(19) United States
(12) Patent Application Publication
(10) Pub. No.: US 2023/0293348 A1
(43) Pub. Date: Sep. 21, 2023

(54) DYES FOR USE IN A METHOD OF PHOTOPORATION OF THE INNER LIMITING MEMBRANE

(71) Applicant: UNIVERSITEIT GENT, Gent (BE)

(72) Inventors: Katrien REMAUT, Gent (BE); Karen PEYNSHAERT, Wondelgem (BE); Stefaan DE SMEDT, Mariakerke (BE); Kevin BRAECKMANS, Daknam (BE)

(73) Assignee: UNIVERSITEIT GENT, Gent (BE)

(21) Appl. No.: 18/014,222

(22) PCT Filed: Jul. 9, 2021
(86) PCT No.: PCT/EP2021/069108

§ 371 Date: Jan. 3, 2023

(30) Foreign Application Priority Data
Jul. 10, 2020 (EP) 20185177.1

(51) Int. Cl. A61F 9/008; A61P 27/02

(52) U.S. Cl. CPC A61F 9/008; A61P 27/02; A61F 2009/00863

(57) ABSTRACT

The invention concerns a dye for use in a method of photoporation of the inner limiting membrane (ILM) of an eye in a subject. Preferably, the method comprises: administering the dye to the vitreous chamber of the eye of the subject; and irradiating at least part of the ILM of the eye of the subject, thereby photoporating at least part of the ILM in the subject. The invention further relates to a dye and optionally a therapeutic agent for use in a method of treating a retinal disease or a choroidal disease of an eye in a subject.

### FIELD OF THE INVENTION

The invention is broadly in the field of medicine, more precisely in the field of ophthalmology. In particular, the invention concerns the use of a dye in a method of photoporation of the inner limiting membrane (ILM) of an eye in a subject.

### BACKGROUND OF THE INVENTION

Today, vision impairment affects approximately 250 million people worldwide of which 36 million are blind. Since many blinding pathologies find their origin in the retina, a vast amount of research is dedicated to the delivery of therapeutic entities (e.g. steroids, antibodies or nucleic acids) to the back of the eye. For mutation-dependent pathologies, gene replacement therapy via sub-retinal injection has made a great leap forward over the last decade. As sub-retinal injections are mainly efficient to reach cells surrounding the injection spot, they are mainly used for treatment of the outer retina, i.e. photoreceptors or retinal pigment epithelium cells (RPE). Nevertheless, the inner retina harbors important target cells as well, including retinal ganglion cells as target for Leber Hereditary Optic Neuropathy (LHON), Müller cells for neurotrophic strategies, and bipolar cells for optogenetic therapy.

The inner limiting membrane (ILM) is an extracellular matrix composed of an intertwined network of collagen type IV and represents the physical border between the vitreous body and the retina. It is well-established that the ILM represents a primary drug delivery barrier for therapeutics delivered after intravitreal injection. Indeed, many retinal therapies or their delivery systems including antibodies cells, viral or non-viral vectors, and nanoparticles are greatly hindered by the ILM and are thus insufficiently reaching the retina in a manner to allow therapy. Notably, the ILM has no essential role in the eye except during embryogenesis and its absence has no effect on the patient’s vision.

Hence, several methods to remove the ILM or to improve transport across the ILM have been proposed in literature. First, it was proven in non-human primates that peeling of the ILM greatly enhanced retinal gene expression by viral vectors. This technique, however, is rather invasive. Prior to ILM peeling, full anesthesia and a vitrectomy is required. While this invasive surgical procedure is performed in the clinic for specific vitreoretinal pathologies such as macular hole or epiretinal membrane, it is never executed for the sake of enhancing retinal delivery. A further method to improve transport across the ILM involves enzymatic digestion of the ILM. It was shown in ex vivo retinal cultures that digestion of the ILM by enzymes enhanced retinal gene expression by viral vectors. However, this was proven to be toxic since the enzymes are not ‘controllable’ and continue digesting the inner retina after digesting the ILM. Recently, an injection technique, called sub-ILM injection, was suggested which should deliver therapeutics right below the ILM and thus within the retina. However, the safety of this technique has not been proven and should be verified further. One publication has attempted to ablate the ILM using an Erbium: yttrium aluminium garnet (YAG) laser in vitro as a solution for macular hole pathology (Hoerauf et al., 2006, Lasers in Surgery and Medicine, 38:52-61). However, it was concluded that defined and reproducible tissue ablation in a range of few micrometers, which is necessary for selective and safe ILM removal, could not be achieved.

Thus, no methods are currently available which allow delivery of therapeutics to the retina in a clinically approved manner. Hence, there remains a need in the art for further and/or improved treatment options for retinal and choroidal disease.

### SUMMARY OF THE INVENTION

The present inventors have unexpectedly found that dyes allow photoporation of the inner limiting membrane, thereby allowing delivery of therapeutic agents to the retina in a minimally invasive manner, hence addressing one or more of the above-mentioned problems in the art.

Accordingly, a first aspect of the invention relates to a dye for use in a method of photoporation of the inner limiting membrane (ILM) of an eye in a subject.

As shown in the experimental section, the present inventors have found that dyes such as vital dyes allow perforation of the ILM in a minimally invasive way. The use of dyes according to the principles illustrating the present invention requires only one injection and the non-invasive application of laser radiation. Moreover, the laser radiation dose applied during treatment can be much lower than the dose currently applied in ophthalmology, hence inducing less or even no retinal toxicity. Furthermore, dyes such as vital dyes approved for clinical use, in particular for ophthalmological use, are commercially available.

Furthermore, the clinical timespan necessary to perform the laser mediated ILM perforation is very short (e.g. laser treatment less than 10 min) which is of equal importance for the ophthalmologist as for the patient. On the contrary, ILM peeling surgery, besides being very invasive, requires more time.

The present use of the dyes such as vital dyes allows delivery of a therapeutic agent to the retina and/or choroid of an eye of a subject. Thereby, the present use of the dyes such as vital dyes allows treatment, such as laser-assisted treatment, of a retinal disease or choroidal disease.

Accordingly, a further aspect relates to a dye such as a vital dye for use in a method of treating a retinal disease or a choroidal disease of an eye in a subject.

A further aspect relates to a dye and a therapeutic agent for use in a method of treating a retinal disease or a choroidal disease of an eye in a subject.

One treatment can have a long-lasting effect since it is established that ILM renewal by the retina is limited and extremely slow. Therefore, the present use of a dye allows repeated delivery of therapeutics. In addition, the present use of a dye for the photoporation of the ILM allows delivery of a wide range of therapeutic agents.

A further aspect provides a method for delivering a therapeutic agent to the retina or the choroid of an eye in a subject, the method comprising:
administering a dye to the vitreous chamber of the eye of the subject;
irradiating at least part of the ILM of the eye of the subject, thereby photoporating at least part of the ILM; and
administering the therapeutic agent to the vitreous chamber of the eye of the subject;
or the method comprising:
administering the dye and the therapeutic agent to the vitreous chamber of an eye of the subject; and
irradiating at least part of the ILM of the eye of the subject, thereby photoporating at least part of the ILM.

The above and further aspects and preferred embodiments of the invention are described in the following sections and in the appended claims. The subject-matter of appended claims is hereby specifically incorporated in this specification.

### DESCRIPTION OF THE DRAWINGS

FIG. 1: Schematic representation of potential mechanism of photoporation concept. ILM: inner limiting membrane; ICG: indocyanine green; VNB: vapour nanobubble; M: Müller cell; G: ganglion cell.

FIG. 2: Images of cryosections of conventional bovine explants stained for nuclei in the retina by Hoechst staining (indicated in grey - low contrast) and showing the ILM stained by antibodies against Collagen IV (indicated in white). A) untreated; B) treatment with 0,4 J/cm² 800 nm 2ps laser pulses; C) treatment with 1 mg/ml ICG and 0,4 J/cm² laser results in ILM ablation, absence of the ILM is represented by the dashed line; Scale = 25 µm.

FIG. 3: SEM imaging of the retinal surface of bovine retinal explants. The smooth surface of untreated explants (left) is greatly disorganized in explants treated with ICG (1 mg/ml) and 0,4 J/cm² laser (right).

FIG. 4: Images of the retinal surface after staining with Mitotracker and Hoechst. The neatly organized mosaic of Müller cell end feet and nerve fibers in untreated explants (left) is greatly disturbed in explants treated with ICG (0,1 mg/ml) and 0,4 J/cm² laser (right). Scale bar = 25 µm.

FIG. 5: Images of cryosections of bovine explants stained for nuclei in the retina by Hoechst (indicated in grey - low contrast). A) 100 nm polystyrene beads (indicated in white) are unable of crossing an intact ILM in untreated explants; B) treatment with 1 mg/ml ICG and 0,4 J/cm² laser leads to high retinal uptake of 100 nm polystyrene beads. Scale = 25 µm

FIG. 6: Images of cryosections of human explants stained for nuclei in the retina by Hoechst staining (indicated in grey - low contrast) and showing the ILM stained by antibodies against Collagen IV (indicated in white); scale = 25 µm. Top row: untreated; bottom row: treatment with 1 mg/ml ICG and 0,4 J/cm² laser fluence. Absence of the ILM is indicated by a dashed line.

FIG. 7: Graph illustrating luciferase mRNA expression detected after 24 hours in the retina of bovine explants treated with mRNA nanoparticles (dark grey) and bovine explants treated with ICG, laser and mRNA nanoparticles (light grey).

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of and from the specified value, in particular variations of +/-10% or less, preferably +/-5% or less, more preferably +/-1% or less, and still more preferably +/-0.1% or less of and from the specified value, insofar such variations are appropriate to perform in the disclosed invention. It is to be understood that the value to which the modifier “about” refers is itself also specifically, and preferably, disclosed.

Whereas the term “one or more”, such as one or more members of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All documents cited in the present specification are hereby incorporated by reference in their entirety.

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention.

By extensive experimental testing, the present inventors have found that dyes are excellent compounds to aid in laser-assisted photoporation of the inner limiting membrane. Dyes such as indocyanine green (ICG) were found not to immobilize at the injection spot but to be sufficiently mobile in the vitreous body to be able to reach and bind the inner limiting membrane. After binding of the dyes to the ILM, irradiation of the ILM with an extremely short laser pulse was found to cause local disruptions of the ILM without causing damage to surrounding cells or tissues like the retina. Contrary to prior Erbium: YAG laser treatment of the ILM, dyes thus allow to create entryways for therapeutics to enter the retina by photoporation of the ILM.

Accordingly, a first aspect of the invention relates to a dye for use in a method of photoporation of the inner limiting membrane (ILM) of an eye in a subject.

Related aspects provide:
a method of photoporation of the ILM of an eye in a subject in need of such a treatment, comprising administering a therapeutically effective amount of a dye to the subject.
the use of a dye for the manufacture of a medicament for the photoporation of the ILM of an eye in a subject.

A further aspect relates to the use of a dye for photoporation of the ILM of an eye of a subject.

The terms “dye” or “stain” as used herein refer to a chemical compound that is capable of binding to various substances in nature to induce colour. Thereby, the dye may increase the visibility of the substance.

As shown in the example section, a dye such as indocyanine green advantageously binds to the ILM, thereby allowing specific accumulation of the dye at the ILM, and functions as a light absorbing agent, thereby allowing the localised destruction of the ILM.

In embodiments of the uses and methods as taught herein, the dye is capable of diffusing in the vitreous chamber (such as in the vitreous body or replacement thereof). In embodiments of the uses and methods as taught herein, the dye is capable of binding to the ILM. In embodiments of the uses and methods as taught herein, the dye is capable of accumulating at the ILM.

In embodiments, the dye is a light absorbing or light sensitizing agent. In embodiments, the dye is capable of absorbing light, such as in the visible light range or near-infrared range. Thus, the radiation used in embodiments of the methods as taught herein may be performed by using a laser emitting laser light in the near-infrared spectrum, e.g. a wavelength of 800 nm. This advantageously lowers interference with surrounding tissues and hence reduces side effects. In embodiments, the irradiation may be performed by using a laser emitting laser light in the visible light spectrum. This advantageously makes the radiation used in the methods as taught herein visible to the clinician, e.g. as opposed to prior art laser treatment that operates outside the visible spectrum.

Accordingly, an aspect provides a dye for use in a method of photoporation of the ILM of an eye in a subject as a light sensitizing agent.

In embodiments of the uses and methods as taught herein, the dye is capable of forming vapour nanobubbles at the ILM when irradiated.

The reference to “a dye” encompasses one or more dyes, such as two or more, three or more, or four or more, such as five, six, seven, eight or more dyes.

The reference to “a dye” encompasses salts thereof, such as pharmaceutically acceptable salts thereof.

A dye may be used on living cells that have been removed from an organism, or may be introduced into the body, e.g. by injection.

In embodiments of the uses and methods as taught herein, the dye may be a biocompatible dye.

In embodiments of the uses and methods as taught herein, the dye may be a vital dye.

The term “vital dye” generally refers to a dye that is capable of binding to living cells or components thereof (such as tissue including the ILM) without inducing immediate evident degenerative changes to the cells or components thereof.

A vital dye may be used on living cells that have been removed from an organism, or may be introduced into the body, e.g. by injection.

In embodiments, the dye such as the vital dye may be a natural dye or a synthetic dye.

In embodiments of the uses and methods as taught herein, the dye, such as the vital dye, may be a dye approved for ophthalmological use. In embodiments, the dye may be a vital dye approved for ophthalmological use.

In embodiments of the uses and methods as taught herein, the dye, such as the vital dye, may be an amphiphilic dye. Such dyes advantageously diffuse through the vitreous and accumulate at the ILM in the eye of a subject.

The term “amphiphilic” refers to the property of possessing both hydrophilic (water-loving, polar) and lipophilic (fat-loving) properties.

In embodiments of the uses and methods as taught herein, the dye such as the vital dye may be selected from the group consisting of an azo dye, an arylmethane dye, a cyanine dye, a thiazine dye, and a xanthene dye.

Examples of azo dyes include Trypan Blue (Membrane Blue, Vision Blue, CAS Number: 72-57-1) and Janus green B (Diazine Green S, Union Green B, CAS Number: 2869-83-2).

Examples of arylmethane dyes include Gentian violet (Crystal violet, Methyl violet 10B, Hexamethyl pararosaniline chloride, CAS Number: 548-62-9); Bromophenol Blue (CAS Number: 115-39-9); Patent blue (Blueron, CAS Number: 3536-49-0); Brilliant Blue (Acid Blue, Coomassie Brilliant Blue, Brilliant Peel, CAS Number: 6104-59-2); Light Green (Light Green SF, Light Green SF Yellowish, CAS Number: 5141-20-8); and Fast Green (Fast Green FCF, Food green 3, FD&C Green No. 3, Green 1724, Solid Green FCF, CAS Number: 2353-45-9).

Examples of cyanine dyes include Indocyanine Green (Cardiogreen, Foxgreen, Cardio-Green, Fox Green, IC Green, CAS Number: 3599-32-4) and Infracyanine Green. Infracyanine Green (IfCG) is a green dye with the same chemical formula and similar pharmacologic properties as ICG. IfCG dye possesses two pharmacologic differences when compared to ICG. First, IfCG contains no sodium iodine, which must be added to ICG during the dye synthesis. Second, the presence of the sodium iodine in the ICG solution necessitates dilution in water, resulting in a hypotonic solution.

Examples of thiazine dyes include Methylene blue (Methylthioninium chloride, CAS Number: 61-73-4) and Toluidine blue (CAS Number: 92-31-9).

Examples of xanthene dyes include Fluorescein Sodium (CAS Number: 518-47-8); Rose Bengal (CAS Number: 4159-77-7); and Rhodamine 6G (Rhodamine 590, Rh6G, C.I. Pigment Red 81, C.I. Pigment Red 169, Basic Rhodamine Yellow, C.I. 45160, CAS Number: 989-38-8).

In embodiments of the uses or methods as taught herein, the dye may be a vital dye selected from the group consisting of: Indocyanine Green (ICG), Trypan Blue (TB), Brilliant Blue (BB), Janus green B (JG), Gentian violet (GV), Bromophenol Blue (BPB), Patent blue (PB), Light Green (LG), Fast Green (FG), Infracyanine Green (IfCG), Methylene blue (MB), Toluidine blue (ToB), Fluorescein Sodium (FS), Rose Bengal (RB), and Rhodamine 6G (R6G). In embodiments, the dye may be Indocyanine Green, Trypan Blue, or Brilliant Blue. Such dyes are advantageously approved for use in ophthalmology.

Preferably, the dye is ICG. ICG is very well suited to generate perforation of the ILM upon illumination with laser light such as pulsed laser light. Moreover, ICG is an FDA-approved dye applied in ophthalmology. ICG advantageously has a wide range of absorbance, thereby allowing the use of near infrared (NIR) radiation which is ideal for in vivo laser treatment. Therefore, ICG allows to tune the wavelength of the laser for instance to NIR, which advantageously lowers the risk of collateral damage to surrounding tissues and cells.

The reference to “a vital dye” encompasses one or more vital dyes, such as two or more, three or more, or four or more, such as five, six, seven, eight or more vital dyes.

The reference to “a vital dye” encompasses salts thereof, such as pharmaceutically acceptable salts thereof.

In embodiments of the uses or methods as taught herein, the dye may be a free or unbound dye, an aggregate of the dye (e.g. H-aggregate or J-aggregate), or a crystal of the dye; or the dye (including an aggregate or crystal thereof) may be conjugated to a further agent (e.g. a polymer, a lipid, a peptide, a protein) and/or the dye (including an aggregate or crystal thereof) may be comprised in a particle, such as a nanoparticle or a microparticle.

The dye such as the vital dye as taught herein can be free dye or can be combined with or chemically bonded to other elements or compounds. In embodiments, the dye may be a free or unbound dye.

Free dyes advantageously allow a localized effect at the ILM. Free dyes allow to specifically bind and efficiently perforate the ILM, even at concentrations which are equal to or below the concentrations used in clinics.

The terms “free” or “unbound” denote that the dye is not combined with or chemically bonded to other elements or compounds, e.g. the dye is not conjugated to another agent, or the dye is not coupled (e.g. grafted) to or enclosed (e.g. encapsulated) in a particle. The free or unbound dyes as taught herein include but are not limited to dyes in solution, and dried or lyophilized dyes, such as a powder of dyes such as a lyophilized powder for injection.

In embodiments of the uses or methods as taught herein, the dye may be an aggregate of the dye (e.g. H-aggregate or J-aggregate) or a crystal of the dye. Such aggregates and crystals advantageously improve the destruction of the ILM because aggregates and dyes remain longer in the vitreous. Due to their larger size, using aggregates or crystals of the dye reduces or avoids entering of the dye into the retina, hence limiting or avoiding retinal toxicity. Further, dye aggregation shifts the absorption wavelength up to higher wavelengths, such as further into the IR region (e.g. 800-900 nm), thereby reducing toxicity as tissues do not or only slightly absorb in this region. In addition, these higher wavelengths, such as in IR region (e.g. 800-900 nm), correspond to the wavelengths of the currently used lasers. Further advantages are the ease of synthesis of the aggregates and the fact they only have the dye in their structure (without any other agent such as polymer or lipid).

The free or unbound dye may be comprised in a composition or formulation such as a pharmaceutical formulation or kit of parts, as will be described further herein. The composition may comprise the dye in a concentration ranging from about 0.001 mg/ml to 5 mg/ml, such as in a concentration of 0.01 ml/ml to 1 mg/ml, or 0.1 mg/ml to 0.5 mg/ml.

In embodiments of the uses or methods as taught herein, the dye (including aggregates or crystals thereof) may be conjugated to a further agent (e.g. a polymer, a lipid, a peptide, a protein) and/or the dye (including aggregates or crystals thereof) may be comprised in a particle, such as a nanoparticle or a microparticle. In embodiments of the uses or methods as taught herein, the dye (including aggregates or crystals thereof) may be grafted on a particle and/or the dye (including aggregates or crystals thereof) may be encapsulated in a particle.

In embodiments of the uses or methods as taught herein, the dye may be conjugated to a further agent and/or the dye may be comprised in a particle such as a nanoparticle or microparticle. The ILM covering the retina has pores which reduce or prevent crossing of large compounds or particles, for instance with a size superior to 100 nm (Peynshaert et al., 2017, Drug Delivery, 24:1, 1384-1394). Hence, conjugating the dye to a further agent and/or comprising the dye in a microparticle or nanoparticle reduces or avoids entering of the dye into the retina, hence limiting or avoiding retinal toxicity.

In embodiments of the uses or methods as taught herein, the dye such as the vital dye may be conjugated to a further agent. The nature of the further agent is not limiting, and the further agent may be any chemical (e.g., inorganic or organic), biochemical or biological substance, molecule or macromolecule (e.g., biological macromolecule).

In embodiments, the further agent may be a polymer, a lipid, a peptide, or a protein.

In embodiments, the polymer may be selected from the group consisting of hyaluronic acid (HA), poly(ethylene) glycol (PEG), poly(DL-lactic-co-glycolic acid) (PLGA), poly(lactic) acid (PLA), polycaprolactone, ethyl cellulose, cellulose acetophthalate, polylactic acid, cellulose, polyvinyl alcohol, polyethylene glycol, gelatine, collagen, silk, alginate, dextran, starch, polycarbonate, polyacrylate, polystyrene, poly(alkyl cyanoacrylate) (PACA), and polyoxazoline. Preferably, the polymer may be hyaluronic acid. For instance, the dye such as ICG may be conjugated to a polymer such as hyaluronic acid. Since the size of the ICG-HA conjugate is larger than the size of the dye itself, administering a conjugate advantageously avoids travel to other parts of the eye and hence reduces or even eliminates toxicity.

In embodiments, the lipid may be anionic, neutral, or cationic lipid. In embodiments, the lipid may be natural, synthetic or bacterial lipid.

Suitable examples of anionic lipids include phosphatidylserine (PS) and phosphatidylglycerol (PG).

Suitable examples of neutral lipids include prostaglandins, eicosanoids, glycerides, glycosylated diacyl glycerols, oxygenated fatty acids, very long chain fatty acids (VLCFA), palmitic acid esters of hydroxystearic acid (PAHSA), N-acylglycine (NAGly), and prenols.

Suitable examples of cationic lipids include multivalent cationic lipids; 1,2-di-O-octadecenyl-3-trimethylammonium propane (DOTMA); ethylphosphocholines (ethyl PC); dimethyldioctadecylammonium (DDAB); pH sensitive lipids; 1,2-dioleoyl-3-trimethylammonium propane (DOTAP); 3β-[N-(N′,N′-dimethylaminoethane)-carbamoyl]cholesterol (DC-Cholesterol); N4-Cholesteryl-spermine (GL67); and 1,2-dioleyloxy-3-dimethylaminopropane (DODMA).

Such lipids are commercially available from Avanti Polar Lipids (Alabama, USA). For instance, a suitable multivalent cationic lipid is (N1-[2-((1S)-1-[(3-aminopropyl)amino]-4-[di(3-amino-propyl)amino]butylcarboxamido)ethyl]-3,4-di[oleyloxy]-benzamide). Examples of ethyl PC include 1,2-dilauroyl-sn-glycero-3-ethylphosphocholine (chloride salt) (12:0 EPC Cl salt); 1,2-dimyristoyl-sn-glycero-3-ethylphosphocholine (chloride salt) (14:0 EPC Cl salt); 1,2-dipalmitoyl-sn-glycero-3-ethylphosphocholine (chloride salt) (16:0 EPC Cl salt); 1,2-distearoyl-sn-glycero-3-ethylphosphocholine (chloride salt) (18:0 EPC Cl salt); 1,2-dioleoyl-sn-glycero-3-ethylphosphocholine (chloride salt) (18:1 EPC Cl salt); 1-palmitoyl-2-oleoyl-sn-glycero-3-ethylphosphocholine (chloride salt) (16:0-18:1 EPC Cl salt); and 1,2-dimyristoleoyl-sn-glycero-3-ethylphosphocholine (Tf salt) (14:1 EPC Tf salt).

Examples of pH sensitive lipids include N-(4-carboxybenzyl)-N,N-dimethyl-2,3-bis(oleoyloxy)propan-1-aminium (DOBAQ); 1,2-distearoyl-3-dimethylammonium-propane (18:0 DAP); 1,2-dipalmitoyl-3-dimethylammonium-propane (16:0 DAP); 1,2-dimyristoyl-3-dimethylammonium-propane (14:0 DAP); 1,2-dioleoyl-3-dimethylammonium-propane (18:1 DAP or DODAP).

In embodiments of the uses and methods as taught herein, the dye such as the vital dye may be comprised in a nanoparticle.

In embodiments, the particle may be a nanosphere or microsphere. The particle may also be a nanorod, a microrod, a nanostar, a microstar, a nanopyramid, a micropyramid, a nanoshell or a microshell. In embodiments, the particle may be a nanosphere. In embodiments, the particle may have a diameter in the range of 1 nm to 1000 nm, for instance 1 nm to 500 nm, e.g. in the range of 50 nm to 500 nm, preferably in the range of 100 nm to 400 nm, or in the range of 100 nm to 350 nm, e.g. in the range of 150 nm to 300 nm. In embodiments, the particle may have a diameter in the range of 5 nm to 300 nm, for instance 10 nm to 250 nm, such as 150 nm to 250 nm.

It is an advantage of particle sizes, e.g. in the range of 100 nm to 350 nm, e.g. in the range of 150 nm to 250 nm, that the mobility of the particles in the vitreous may be improved.

In embodiments, the nanoparticles, in particular for instance their core, may comprise a polymer material, carbon and/or titanium. The core may comprise melanin. The core may comprise poly-dihydroxyphenylalanine (DOPA).

The nanoparticle may be a polymer nanoparticle, a protein nanoparticle or lipid nanoparticle (i.e. liposome). The polymer may be poly(lactic-co-glycolic acid) (PLGA). For example, PLGA-based ICG nanoparticles (PLGA-ICG NPs) may be prepared as described in Saxena et al., 2004, Int J Pharm, 278(2):293-301. The protein may be human serum albumin (HSA). For example, human serum albumin ICG nanoparticles (HSA-ICG NPs) may be prepared as described in Sheng et al., 2014, ACS Nano, 8(12):12310-22. The lipid nanoparticle may be MC3-based lipid nanoparticles (Patel et al., 2019, J. Control. Release, 303, 91-100). Liposomes encapsulating ICG (Lip-ICG) may be prepared as described in Lajunen et al., 2018, J. Control. Release 284, 213-223.

The particles, e.g. the particle size, may be characterized by dynamic light scattering (DLS), transmission electron microscopy (TEM), UV-vis spectroscopy, and/or electrodynamic modeling using Mie theory. The obtained concentration of the particles may be estimated using experimental extinction intensities at the maximum wavelength, and Mie theory calculations of the extinction cross section for spherical particles. The encapsulation efficiency (i.e. dye loading efficiency) may be determined based on a calibration curve of free dye obtained by UV-vis spectrometry or fluorescence. The zeta-potential may be measured by electrophoretic mobility.

In embodiments of the uses and methods as taught herein, the dye may be grafted on a particle, such as a nanoparticle or a microparticle. In embodiments, the dye may be grafted on a nanoparticle. For instance, the dye may be grafted on a particle by ‘click-chemistry’ at the surface of the particle (e.g. at the end of polymer chains such as at the distal end of poly(ethylene) glycol chains or hyaluronic acid chains). For instance, the grafting of a dye may occur at the end of PEG chains that are grafted on the particles.

In embodiments of the uses and methods as taught herein, the dye may be encapsulated in a particle, such as a nanoparticle or a microparticle. In embodiments, the dye may be encapsulated in a nanoparticle. In embodiments, the dye may be encapsulated in a particle by physical or chemical encapsulation. For instance, physical encapsulation of ICG in liposomes may be performed by adding ICG during the rehydration of the lipids. For HAS-ICG particles, the chemical encapsulation may be performed by reacting ICG with the disulphide bonds of HAS.

The dye as taught herein advantageously allows efficient and targeted photoporation of the ILM.

The term “photoporation” as used herein refers to the process of using electromagnetic radiation, such as (visible) light or near-infrared radiation, to create pores in a cell membrane. The electromagnetic radiation may be generated by a laser such as a pulsed laser.

In embodiments, the photoporation may be laser-assisted photoporation.

The use of the dye as taught herein may lead to fragmentation or even destruction of the ILM. Hence, further aspects or embodiments relate to the dye as taught herein for use in a method of photodestruction of the ILM.

The terms “photodestruction” as used herein refers to a process of using electromagnetic radiation, such as (visible) light or near-infrared radiation, to remove at least part of a cell membrane.

In embodiments, the photodestruction may be laser-assisted photodestruction.

The term “inner limiting membrane (ILM)” as used herein refers to a layer of the retina (i.e. retinal layer) which forms the boundary between the vitreous body and (the other layers of) the retina. The ILM comprises a basement membrane and aligns with the endfeet of Müller cells.

In embodiments, the ILM may have a thickness of about 100 nm to about 5 µm, e.g. of about 100 nm to about 4 µm. In embodiments, when the subject is a human subject, the ILM may have an average thickness of about 0.5 µm to about 5 µm, such as of about 1 µm to about 4 µm or of about 1.5 µm to about 4 µm. The foveal pit located at the centre of the human macula exhibits a thickness of about 100 nm.

The terms “vitreous body”, “vitreous humour” or “the vitreous” can be used interchangeably herein and refer to a clear gel that fills the space between the lens and the retina of the eyeball of humans and other vertebrates. The vitreous body contains water (e.g. 98-99% of its volume is water) and a network consisting of collagen and glycosaminoglycans such as hyaluronic acid (HA).

The term “retina” as used herein refers to the light-sensitive layer of tissue of the eye. The vertebrate retina has distinct layers (from closest to farthest from the vitreous body): ILM (basement membrane elaborated by Müller cells); Nerve fiber layer (axons of the ganglion cell bodies); Ganglion cell layer (contains nuclei of ganglion cells, the axons of which become the optic nerve fibers, and some displaced amacrine cells); Inner plexiform layer (contains the synapse between the bipolar cell axons and the dendrites of the ganglion and amacrine cells); Inner nuclear layer (contains the nuclei and surrounding cell bodies of the amacrine cells, bipolar cells, and horizontal cells); Outer plexiform layer (projections of rods and cones ending in the rod spherule and cone pedicle, respectively. In the macular region, this is known as the Fiber layer of Henle); Outer nuclear layer (cell bodies of rods and cones); External limiting membrane (layer that separates the inner segment portions of the photoreceptors from their cell nuclei); Inner segment / outer segment layer (inner segments and outer segments of rods and cones); Retinal pigment epithelium (single layer of cuboidal epithelial cells; this layer is closest to the choroid).

The terms “choroid”, “choroidea” or “choroid coat” as used herein refers to the vascular layer of the eye. The choroid lies between the retina and the sclera. The human choroid is thickest at the far extreme rear of the eye (at 0.2 mm), while in the outlying areas it narrows to 0.1 mm. The choroid provides oxygen and nourishment to the outer layers of the retina.

The term “eye” as used herein has its meaning as ordinary in the art and refers to the organs of the visual system.

The terms “subject”, “individual” or “patient” can be used interchangeably herein, and typically and preferably denote humans, but may also encompass reference to non-human animals, preferably warm-blooded animals, even more preferably mammals, such as, e.g., non-human primates, rodents, canines, felines, equines, ovines, porcines, and the like. The term “non-human animals” includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In certain embodiments, the subject is a non-human mammal. In certain embodiments, the subject is a human subject. The term does not denote a particular age or sex. Thus, adult and newborn subjects, as well as fetuses, whether male or female, are intended to be covered. Examples of subjects include humans, dogs, cats, cows, goats, and mice. The term subject is further intended to include transgenic species.

Suitable subjects may include without limitation subjects presenting to a physician for a screening for a retinal disease or a choroidal disease, subjects presenting to a physician with symptoms and signs indicative of a retinal disease or a choroidal disease, subjects diagnosed with a retinal disease or a choroidal disease, and subjects who have received an alternative (unsuccessful) treatment for a retinal disease or a choroidal disease.

In embodiments of the uses or methods as taught herein, the method may comprise:
administering the dye to the vitreous chamber of the eye of the subject; and
irradiating at least part of the ILM of the eye of the subject.

Thereby, the dye advantageously allows forming perforations in the ILM.

In embodiments of the uses or methods as taught herein, the method may comprise:
administering the dye to the vitreous chamber of the eye of the subject; and
irradiating at least part of the ILM of the eye of the subject, thereby photoporating at least part of the ILM in the subject.

Hence, an aspect provides a dye for use in a method of photoporation of the inner limiting membrane (ILM) of an eye in a subject, wherein the method comprises:
administering the dye to the vitreous chamber of the eye of the subject; and
irradiating at least part of the ILM of the eye of the subject, thereby photoporating at least part of the ILM in the subject.

A related aspect provides a method of photoporation of the inner limiting membrane (ILM) of an eye in a subject, wherein the method comprises:
administering a dye to the vitreous chamber of the eye of the subject; and
irradiating at least part of the ILM of the eye of the subject, thereby photoporating at least part of the ILM in the subject.

In embodiments, the method comprises administering the dye to the vitreous chamber of an eye of the subject. In embodiments of the uses and methods as taught herein, the dye may be administered to the vitreous chamber by injection.

In embodiments, the dye such as the vital dye may be administered to the vitreous chamber comprising the vitreous body. Hence, in embodiments, the dye such as the vital dye may be administered to the vitreous body of the eye of the subject. In embodiments of the uses and methods as taught herein, the dye such as the vital dye may be administered to the vitreous chamber by intravitreal administration. Intravitreal administration advantageously allows delivery of the dye directly to the vitreous body. In embodiments of the uses and methods as taught herein, the dye such as the vital dye may be administered to the vitreous chamber by intravitreal injection. Intravitreal injection allows delivery of the dye directly to the vitreous body by a minimally invasive technique, thereby reducing the risks and pain for the patient and increasing the patient’s well-being.

The term “intravitreal administration” as used herein refers to a process or procedure to place a medication (e.g. the dye and/or the therapeutic agent, or the composition as taught herein) directly into the vitreous cavity which is filled with the vitreous body.

In embodiments, the dye such as the vital dye may be administered to the vitreous chamber after removal of the vitreous body (i.e. after vitrectomy). Thus, in embodiments, the dye such as the vital dye may be administered to the vitreous chamber no longer comprising the vitreous body. In embodiments of the uses or methods as taught herein, the dye such as the vital dye may be administered by injection into the vitreous chamber after removal of the vitreous body.

After removal of the vitreous, the vitreous body may be replaced by a replacement (i.e. substitute) of the vitreous body such as a Balanced Salt Solution (BSS), gas (e.g. perfluoropropane), or silicone oil. After vitrectomy, the vitreous may be briefly exchanged by air, after which the dye can be flushed through the eye followed by filling the eye with the replacement (i.e. substitute) of the vitreous, such as a BSS, gas (e.g. perfluoropropane), or silicone oil. Or after replacement of the vitreous, the dye can be administered into the replacement (i.e. substitute) of the vitreous, such as a BSS, gas (e.g. perfluoropropane), or silicone oil. In embodiments, the dye may be injected into the replacement of the vitreous body, e.g. into the BSS, gas, or silicone oil, after removal of the vitreous body.

In embodiments of the uses and methods as taught herein, the dye such as the vital dye may be administered at a concentration of about 0.001 mg/ml to about 5.0 mg/ml. In embodiments, the dye such as the vital dye may be administered at a concentration of about 0.01 mg/ml to about 1.0 mg/ml. In embodiments, the dye such as the vital dye may be administered at a concentration of about 0.1 mg/ml to about 0.5 mg/ml. In embodiments of the uses or methods as taught herein, the dye such as the vital dye may be administered at a concentration of about 0.001 mg/ml to about 0.5 mg/ml. Such concentration is equal or lower than the concentration of a dye typically used in the clinic (e.g. typically used concentration in the clinic, in particular in ophthalmology, of for example TB is 0.6 mg/ml and of ICG is 1.25 mg/ml). The concentration advantageously allows treatment without any toxicity to the surrounding ocular tissues.

In embodiments, when using ICG as the dye, the dye may be administered at a concentration of at least 0.01 mg/ml, such as at least 0.1 mg/ml, preferably at a concentration of at least 0.5 mg/ml. For instance, when using ICG as the dye, the dye may be administered at a concentration of about 0.001 mg/ml to about 1.0 mg/ml, such as at a concentration of about 0.001 mg/ml to about 0.5 mg/ml, or at a concentration of about 0.01 mg/ml to about 0.5 mg/ml, preferably at a concentration of about 0.1 mg/ml to about 0.5 mg/ml. Such concentration is lower than the current clinically used concentration and allows treatment without any toxicity to the surrounding ocular tissues.

In embodiments of the uses and methods as taught herein, the dye such as the vital dye may be capable of diffusing in the vitreous chamber (such as in the vitreous body or replacement thereof) after administration, for instance by intravitreal injection. When using the dye such as the vital dye in accordance with embodiments of the invention, the dye may diffuse in the vitreous chamber (such as in the vitreous body or replacement thereof) after administration.

In embodiments of the uses and methods as taught herein, the dye such as the vital dye may be capable of binding to (accumulating at) the ILM after administration, for instance by intravitreal injection. When using the dye such as the vital dye in accordance with embodiments of the invention, the dye may bind to (accumulate at) the ILM after administration.

The binding of the dye to the ILM may be binding by a covalent binding or a non-covalent interaction.

In embodiments, the method may comprise administering the dye to the vitreous body of an affected eye of the subject, thereby inducing binding of the dye to (accumulation of the dye at) the ILM. In embodiments, the method may comprise administering the dye to the vitreous body of an affected eye of the subject, thereby inducing diffusion of the dye in the vitreous chamber (such as in the vitreous body or replacement thereof) and binding of the dye to (accumulation of the dye at) the ILM.

In embodiments of the uses and methods as taught herein, the dye such as the vital dye may be capable of forming vapor nanobubbles at the ILM when irradiated. When using the dye such as the vital dye in accordance with embodiments of the invention, the dye may form vapor nanobubbles at the ILM when irradiated.

In embodiments, the method may comprise irradiating the dye bound to at least part of the ILM, thereby photoporating at least part of the ILM in the subject. In embodiments, the method may comprise irradiating the dye bound to at least part of the ILM, thereby forming vapour nanobubbles at the ILM and photoporating at least part of the ILM in the subject.

In embodiments, the method may comprise:
administering the dye to the vitreous chamber of the eye of the subject, thereby inducing binding of the dye to (accumulation of the dye at) the ILM; and
irradiating the dye bound to (accumulated at) at least part of the ILM of the eye of the subject, thereby photoporating at least part of the ILM in the subject.

In embodiments of the uses and methods as taught herein, the dye such as the vital dye is capable of diffusing in the vitreous chamber (such as in the vitreous body or replacement thereof). When using the dye such as the vital dye in accordance with embodiments of the invention, the dye may diffuse in the vitreous chamber (such as in the vitreous body or replacement thereof) after administration.

When using the dye such as the vital dye in accordance with embodiments of the invention, the dye such as the vital dye may accumulate at the ILM after administration. In embodiments of the uses and methods as taught herein, the dye such as the vital dye is capable of accumulating at the ILM.

In embodiments of the uses and methods as taught herein, the dye such as the vital dye is capable of forming vapour nanobubbles at the ILM when irradiated. When using the dye such as the vital dye in accordance with embodiments of the invention, the dye such as the vital dye may form vapour nanobubbles at the ILM when irradiated.

In embodiments of the uses and methods as taught herein, the method may comprise irradiating at least part of the ILM of the eye of the subject with radiation, thereby photoporating at least part of the ILM in the subject.

In embodiments of the uses and methods as taught herein, the at least part of the ILM may be irradiated with electromagnetic radiation.

The terms “radiation” and “electromagnetic radiation” may be used interchangeably herein.

In embodiments, the electromagnetic radiation is infrared radiation (including near infrared) or visible light.

In embodiments, the at least part of the ILM may be irradiated with laser radiation. In embodiments, the at least part of the ILM may be irradiated with pulsed-laser radiation. Laser irradiation, such as irradiation by pulsed lasers, e.g. pico-, femto- and/or nanosecond pulsed lasers, can be combined with a dye in accordance with embodiments of the present invention to efficiently destroy the ILM, e.g. by laser-induced vapor nanobubble generation. While laser irradiation may be advantageous, irradiation by another (intense) light source is not necessarily excluded to achieve the same or similar effects.

In embodiments of the uses or methods as taught herein,
the intensity of the pulses of the laser may be at least 10⁴ W/cm²;
the number of pulses of the laser may be at least 1 laser pulse; and/or
the pulses of the laser may have a duration in the range of at least 10 fs.

In embodiments of the uses or methods as taught herein, the laser irradiation reaches a fluence of at least 0.1 mJ/cm² at the ILM.

In embodiments of the uses or methods as taught herein,
the intensity of the pulses of the laser may be 10⁴ to 10¹⁵ W/cm² or 10⁷ to 10¹⁵ W/cm²;
the number of pulses of the laser may be 1 to 1000 laser pulses; and/or
the pulses of the laser may have a duration in the range of 10 fs to 10 ns.

In embodiments, the intensity of the pulses of the laser may be 10⁷ to 10¹⁵ W/cm², such as in the range of 10¹² to 10¹⁴ W/cm². The laser pulses may each have a power density or intensity in the range of 10⁷ to 10¹⁵ W/cm², e.g. in the range of 10¹² to 10¹⁵ W/cm², or alternatively expressed, a fluence in the range of 10 µJ/cm² to 100 J/cm², e.g. in the range of 10 mJ/cm² to 10 J/cm² or in the range of 1 J/cm² to 10 J/cm².

In embodiments, the number of pulses of the laser may be 1 to 1000 laser pulses; such as 1 to 500 laser pulses, 1 to 100 laser pulses, 1 to 20 laser pulses, or 1 to 10 laser pulses. The number of laser pulses may be depending on various factors such as on the thickness of the ILM, the desired pore size and/or degree of destruction of the ILM.

In embodiments, the pulses of the laser may have a duration in the range of 10 fs to 10 ns, for instance in the range of 10 fs to 1 ps or in the range of 1 ps to 10 ns.

In embodiments, the dye such as the vital dye is a light absorbing or light sensitizing agent. In embodiments, the dye such as the vital dye is capable of absorbing light, such as in the visible light range or near-infrared range.

The dye such as the vital dye as taught herein may be used as a light sensitizing agent in the uses or methods as taught herein. In embodiments, the dye such as the vital dye is used as a light sensitizing agent.

Hence, aspects relate to:
A dye such as a vital dye for use in a method of photoporation of the ILM (of an eye) in a subject, wherein the dye such as the vital dye is used as a light sensitizing agent.
A dye such as a vital dye for use in a method of treatment, such as laser-assisted treatment or phototherapy, of a retinal disease or a choroidal disease (of an eye) in a subject, wherein the dye such as the vital dye is used as a light sensitizing agent.
A dye such as a vital dye and a therapeutic agent for use in a method of treatment, such as laser-assisted treatment or phototherapy, of a retinal disease or a choroidal disease (of an eye) in a subject, wherein the dye such as the vital dye is used as a light sensitizing agent.

The uses or methods as taught herein may comprise injecting the dye such as the vital dye into the vitreous chamber (e.g. comprising the vitreous body or replacement thereof) of an eye of a human or animal subject. The uses or methods as taught herein may comprise a pulsed-laser radiation after injection of the dye such as the vital dye into the vitreous chamber of an eye of a human or animal subject.

When using the dye such as the vital dye in accordance with embodiments of the invention, the dye such as the vital dye may specifically bind to the ILM and may locally exert a mechanical force onto the ILM when irradiated e.g. by pulsed-laser radiation.

When using the dye such as the vital dye in accordance with embodiments of the invention, the dye such as the vital dye may form vapor nanobubbles in the vitreous chamber (e.g. comprising the vitreous body or a replacement thereof) when being irradiated, so as to exert a mechanical force onto the ILM.

When using the dye such as the vital dye in accordance with embodiments of the invention as a light sensitizing agent in the uses or methods as taught herein, the dye such as the vital dye may cluster around the ILM to concentrate energy deposition by the radiation near and/or in the ILM, such that a collapse of the vapor nanobubbles releases a mechanical force to form perforations; and/or to dislodge and/or break apart the ILM.

The present use of the dyes such as the vital dyes allows delivery of a therapeutic agent to the retina and/or choroid of an eye of a subject. Thereby, the present use of the dyes such as the vital dyes allows treatment, such as laser-assisted treatment or phototherapy, of a retinal or choroidal disease.

A further aspect relates to a dye for use in a method of treatment of a retinal disease or a choroidal disease in a subject.

Related aspects provide:
a method of treatment of a retinal disease or a choroidal disease in a subject in need of such a treatment, comprising administering a therapeutically effective amount of a dye to the subject.
use of a dye for the manufacture of a medicament for the treatment of a retinal disease or a choroidal disease.
the use of a dye for the treatment of a retinal disease or a choroidal disease.

A further aspect provides a dye and a therapeutic agent for use in a method of treatment of a retinal disease or a choroidal disease in a subject.

Related aspects provide:
a method of treatment of a retinal disease or a choroidal disease in a subject in need of such a treatment, comprising administering a therapeutically effective amount of a dye and a therapeutic agent to the subject.
use of a dye and a therapeutic agent for the manufacture of a medicament for the treatment of a retinal disease or a choroidal disease.
the use of a dye and a therapeutic agent for the treatment of a retinal disease or a choroidal disease.

Preferably, an aspect provides a dye and a therapeutic agent for use in a method of treating a retinal disease or a choroidal disease of an eye in a subject, wherein the method comprises:
administering the dye to the vitreous chamber of the eye of the subject;
irradiating at least part of the ILM of the eye of the subject, thereby photoporating at least part of the ILM; and
administering the therapeutic agent to the vitreous chamber of the eye of the subject, thereby treating the retinal disease or the choroidal disease in the subject;
or wherein the method comprises:
administering the dye and the therapeutic agent to the vitreous chamber of an eye of the subject; and
irradiating at least part of the ILM of the eye of the subject, thereby photoporating at least part of the ILM and treating the retinal disease or the choroidal disease in the subject.

A related aspect provides a method of treating a retinal disease or a choroidal disease of an eye in a subject, wherein the method comprises:
administering a dye to the vitreous chamber of the eye of the subject;
irradiating at least part of the ILM of the eye of the subject, thereby photoporating at least part of the ILM; and
administering a therapeutic agent to the vitreous chamber of the eye of the subject, thereby treating the retinal disease or the choroidal disease in the subject;
or wherein the method comprises:
administering a dye and a therapeutic agent to the vitreous chamber of an eye of the subject; and
irradiating at least part of the ILM of the eye of the subject, thereby photoporating at least part of the ILM and treating the retinal disease or the choroidal disease in the subject.

In embodiments, the retinal or choroidal diseases may be selected from the group consisting of inherited retinal dystrophies (such as choroidal dystrophy, choroideremia, familial exudative vitreoretinopathy, Leber Congenital Amaurosis, Usher Syndrome, Stargardt Disease, Cone Dystrophy, Cone Rod Dystrophy, Achromatopsia, occult macular dystrophy, retinitis pigmentosa, retinoschisis, Bietti’s crystalline dystrophy and vitelliform macular dystrophy); acquired diseases of the retina or choroid (such as central serous chorioretinopathy, central serous retinopathy, diabetic retinopathy, epiretinal membrane, geography atrophy, glaucoma, macular degeneration, macular edema and macular scarring); inflammatory diseases of the retina or choroid (such as acute retinal necrosis, autoimmune retinopathy, choroiditis, chorioretinitis, choroidopathy, cytomegalovirus retinitis, diffuse unilateral subacute neuroretinitis, multifocal choroiditis, panuveitis, uveitis, multiple evanescent white dot syndrome, punctate inner choroiditis, retinopathy of prematurity, toxoplasmic chorioretinitis); vascular diseases of the retina or choroid (such as branch retinal artery occlusion, branch retinal vein occlusion, central artery occlusion, central retinal vein occlusion, choroidal neovascularization, retinal neovascularization, Coat’s disease, hypertensive retinopathy, ocular ischemic syndrome, polypoidal choroidal vasculopathy, retinal haemorrhage, retinopathy); neoplastic diseases of the retina or choroid (such as choroidal melanoma, retinoblastoma); ocular traumas (such as Berlin’s edema, Purtscher’s retinopathy, radioation retinopathy, choroidal rupture); retinal tractional defects (such as retinal tear, macular hole, retinal detachment, choroidal detachment); and toxic retinopathy (such as chloroquine retinopathy).

In embodiments, the retinal or choroidal disease may be selected from the group consisting of acute retinal necrosis, ametropic amblyopia, autoimmune retinopathy, Berlin’s edema, Bietti’s crystalline dystrophy, birdshot chorioretinopathy, branch retinal artery occlusion, branch retinal vein occlusion, central retinal artery occlusion, central retinal vein occlusion, central serous chorioretinopathy, central serous retinopathy, chloroquine retinopathy, choroidal detachment, choroidal dystrophies, choroidal folds, choroidal melanoma, choroidal neovascularization, choroidal rupture, choroiditis, chorioretinitis, choroideremia, choroidopathy, Coats’ disease, cytomegalovirus retinitis, diabetic retinopathy, diffuse unilateral subacute neuroretinitis, epiretinal membrane, familial exudative vitreoretinopathy, geographic atrophy, glaucoma, hypertensive retinopathy, Inherited Retinal Dystrophies (IRD’s, e.g. Leber Congenital Amaurosis), macular degeneration, macular edema, macular hole, macular scarring, multifocal choroiditis and panuveitis, multiple evanescent white dot syndrome, occult macular dystrophy, ocular ischemic syndrome, polypoidal choroidal vasculopathy, progressive retinal atrophy, punctate inner choroiditis, Purtscher’s retinopathy, radiation retinopathy, retinal detachment, retinal haemorrhage, retinal tear, retinitis, retinitis pigmentosa, retinoblastoma, retinopathy, retinopathy of prematurity, retinoschisis, toxoplasmic chorioretinitis, and vitelliform macular dystrophy.

The term “retinal disease” as used herein refers to any disease or disorder that affects at least part of the retina.

In embodiments, the retinal disease may be selected from the group consisting of macular degeneration, diabetic retinopathy, retinitis pigmentosa, glaucoma, retinoblastoma, and Inherited Retinal Dystrophies.

The term “choroidal disease” as used herein refers to any disease or disorder that affects at least part of the choroid.

In embodiments, the choroidal disease may be selected from the group consisting of central serous chorioretinopathy, polypoidal choroidal vasculopathy, choroidal melanoma, choroidal neovascularization, choroideremia, and choroidal dystrophies.

In embodiments, the dye as taught herein and the therapeutic agent as taught herein may be dosed independently, i.e. are present in a kit of parts in different unit doses or dosage forms. Said separate dosage forms can be administered simultaneously and/or at different time points, such as chronologically staggered, that is at different time points. The dye as taught herein and the therapeutic agent as taught herein can be administered by the same route or by different routes.

In embodiments, the dye as taught herein and the therapeutic agent as taught herein may be administered simultaneously or sequentially.

In embodiments, the dye as taught herein and the therapeutic agent as taught herein may be administered separately and sequentially.

In embodiments, the dye may be administrated before the therapeutic agent. In embodiments, the dye may be administrated at least 5 min before treatment with the therapeutic agent is started. In embodiments, the dye may be administrated at least 10 min before, such as at least 15 min, at least 30 min before, at least 1 hour before, at least 2 hours before, at least 3 hours before, at least 4 hours before, or at least 6 hours before, treatment with the therapeutic agent.

Hence, in embodiments of the uses or methods as taught herein, the method may comprise:
administering the dye to the vitreous chamber of the eye of the subject;
irradiating at least part of the ILM of the eye of the subject, thereby photoporating at least part of the ILM; and
administering the therapeutic agent to the vitreous chamber of the eye of the subject, thereby treating the retinal disease or the choroidal disease in the subject.

In embodiments, the dye as taught herein and the therapeutic agent as taught herein may be administered simultaneously.

Hence, in embodiments of the uses or methods as taught herein, the method may comprise:
administering the dye and the therapeutic agent to the vitreous chamber of an eye of the subject; and
irradiating at least part of the ILM of the eye of the subject, thereby photoporating at least part of the ILM and treating the retinal disease or the choroidal disease in the subject.

In embodiments, the dye as taught herein and the therapeutic agent as taught herein may be comprised in a composition for simultaneous administration or in a kit of parts for simultaneous or sequential administration.

As used herein, the term “agent” broadly refers to any chemical (e.g., inorganic or organic), biochemical or biological substance, molecule or macromolecule (e.g., biological macromolecule), a combination or mixture thereof (e.g. cell), a sample of undetermined composition, or an extract made from biological materials such as bacteria, plants, fungi, or animal cells or tissues. Preferred though non-limiting “agents” include nucleic acids, oligonucleotides, ribozymes, peptides, polypeptides, proteins, peptidomimetics, antibodies, antibody fragments, antibody-like protein scaffolds, aptamers, photoaptamers, spiegelmers, chemical substances, preferably organic molecules, more preferably small organic molecules, lipids, carbohydrates, polysaccharides, etc., and any combinations thereof. Depending on the context, the term “agent” may denote a “therapeutic agent” or “drug”, useful for or used in the treatment, cure, prevention, or diagnosis of a disease.

In embodiments of the uses or methods as taught herein, the therapeutic agent may be selected from the group consisting a stem cell, a protein, a polypeptide, a peptide, an antibody, an antibody fragment, an antibody-like protein scaffold, an aptamer, a photoaptamer, a spiegelmer, a peptidomimetic, a gene-editing system, a nucleic acid (including oligonucleotides), and combinations thereof.

Stem cells in ophthalmology can potentially serve two different therapeutic roles: regenerative or trophic. For example, stem cells have the potential to replace or regenerate tissue, such as retinal ganglion cells in glaucoma, or retinal pigment epithelium in retinitis pigmentosa or age-related macular degeneration (AMD)-related geographic atrophy (GA). They can alternatively or simultaneously assume a trophic role, producing growth factors and cytokines, such as brain-derived neurotrophic factor, that have a supportive paracrine effect on local structures within the macula. The stem cells, in particular somatic stem cells such as mesenchymal stem cells, are typically delivered by intravitreal administration.

The term “protein” as used herein generally encompasses macromolecules comprising one or more polypeptide chains, i.e., polymeric chains of amino acid residues linked by peptide bonds. The term may encompass naturally, recombinantly, semi-synthetically or synthetically produced proteins. The term also encompasses proteins that carry one or more co- or post-expression-type modifications of the polypeptide chain(s), such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes protein variants or mutants which carry amino acid sequence variations vis-à-vis a corresponding native protein, such as, e.g., amino acid deletions, additions and/or substitutions. The term contemplates both full-length proteins and protein parts or fragments, e.g., naturally occurring protein parts that ensue from processing of such full-length proteins.

The term “polypeptide” as used herein encompasses polymeric chains of amino acid residues linked by peptide bonds. Hence, especially when a protein is only composed of a single polypeptide chain, the terms “protein” and “polypeptide” may be used interchangeably herein to denote such a protein. The term is not limited to any minimum length of the polypeptide chain. The term may encompass naturally, recombinantly, semi-synthetically or synthetically produced polypeptides. The term also encompasses polypeptides that carry one or more co- or post-expression-type modifications of the polypeptide chain, such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes polypeptide variants or mutants which carry amino acid sequence variations vis-à-vis a corresponding native polypeptide, such as, e.g., amino acid deletions, additions and/or substitutions. The term contemplates both full-length polypeptides and polypeptide parts or fragments, e.g., naturally occurring polypeptide parts that ensue from processing of such full-length polypeptides.

The term “peptide” as used throughout this specification preferably refers to a polypeptide as used herein consisting essentially of 50 amino acids or less, e.g., 45 amino acids or less, preferably 40 amino acids or less, e.g., 35 amino acids or less, more preferably 30 amino acids or less, e.g., 25 or less, 20 or less, 15 or less, 10 or less or 5 or less amino acids.

As used herein, the term “antibody” is used in its broadest sense and generally refers to any immunologic binding agent. The term specifically encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest, i.e., antigen-binding fragments), as well as multivalent and/or multi-specific composites of such fragments. The term “antibody” is not only inclusive of antibodies generated by methods comprising immunisation, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro or in vivo.

An antibody may be any of IgA, IgD, IgE, IgG and IgM classes, and preferably IgG class antibody. An antibody may be a polyclonal antibody, e.g., an antiserum or immunoglobulins purified there from (e.g., affinity-purified). An antibody may be a monoclonal antibody or a mixture of monoclonal antibodies. Monoclonal antibodies can target a particular antigen or a particular epitope within an antigen with greater selectivity and reproducibility. By means of example and not limitation, monoclonal antibodies may be made by the hybridoma method first described by Kohler et al. 1975 (Nature 256: 495), or may be made by recombinant DNA methods (e.g., as in US 4,816,567). Monoclonal antibodies may also be isolated from phage antibody libraries using techniques as described by Clackson et al. 1991 (Nature 352: 624-628) and Marks et al. 1991 (J Mol Biol 222: 581-597), for example.

Antibody binding agents may be antibody fragments. “Antibody fragments” comprise a portion of an intact antibody, comprising the antigen-binding or variable region thereof. Examples of antibody fragments include Fab, Fab′, F(ab′)2, Fv and scFv fragments, single domain (sd) Fv, such as VH domains, VL domains and VHH domains; diabodies; linear antibodies; single-chain antibody molecules, in particular heavy-chain antibodies; and multivalent and/or multispecific antibodies formed from antibody fragment(s), e.g., dibodies, tribodies, and multibodies. The above designations Fab, Fab′, F(ab′)2, Fv, scFv etc. are intended to have their art-established meaning.

The term antibody includes antibodies originating from or comprising one or more portions derived from any animal species, preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel (e.g., Camelus bactrianus and Camelus dromaderius), llama (e.g., Lama paccos, Lama glama or Lama vicugna) or horse.

A skilled person will understand that an antibody can include one or more amino acid deletions, additions and/or substitutions (e.g., conservative substitutions), insofar such alterations preserve its binding of the respective antigen. An antibody may also include one or more native or artificial modifications of its constituent amino acid residues (e.g., glycosylation, etc.).

Methods of producing polyclonal and monoclonal antibodies as well as fragments thereof are well known in the art, as are methods to produce recombinant antibodies or fragments thereof (see for example, Harlow and Lane, “Antibodies: A Laboratory Manual”, Cold Spring Harbour Laboratory, New York, 1988; Harlow and Lane, “Using Antibodies: A Laboratory Manual”, Cold Spring Harbour Laboratory, New York, 1999, ISBN 0879695447; “Monoclonal Antibodies: A Manual of Techniques”, by Zola, ed., CRC Press 1987, ISBN 0849364760; “Monoclonal Antibodies: A Practical Approach”, by Dean & Shepherd, eds., Oxford University Press 2000, ISBN 0199637229; Methods in Molecular Biology, vol. 248: “Antibody Engineering: Methods and Protocols”, Lo, ed., Humana Press 2004, ISBN 1588290921).

In certain embodiments, the agent may be a Nanobody®. The terms “Nanobody®” and “Nanobodies®” are trademarks of Ablynx NV (Belgium). The term “Nanobody” is well-known in the art and as used herein in its broadest sense encompasses an immunological binding agent obtained (1) by isolating the V_{HH} domain of a heavy-chain antibody, preferably a heavy-chain antibody derived from camelids; (2) by expression of a nucleotide sequence encoding a V_{HH} domain; (3) by “humanization” of a naturally occurring V_{HH} domain or by expression of a nucleic acid encoding a such humanized V_{HH} domain; (4) by “camelization” of a V_{H} domain from any animal species, and in particular from a mammalian species, such as from a human being, or by expression of a nucleic acid encoding such a camelized V_{H} domain; (5) by “camelization” of a “domain antibody” or “dAb” as described in the art, or by expression of a nucleic acid encoding such a camelized dAb; (6) by using synthetic or semi-synthetic techniques for preparing proteins, polypeptides or other amino acid sequences known per se; (7) by preparing a nucleic acid encoding a Nanobody using techniques for nucleic acid synthesis known per se, followed by expression of the nucleic acid thus obtained; and/or (8) by any combination of one or more of the foregoing. “Camelids” as used herein comprise old world camelids (Camelus bactrianus and Camelus dromaderius) and new world camelids (for example Lama paccos, Lama glama and Lama vicugna).

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat). Such scaffolds have been extensively reviewed in Binz et al., Gebauer and Skerra, Gill and Damle, Skerra 2000, and Skerra 2007, and include without limitation affibodies, based on the Z-domain of staphylococcal protein A, a three-helix bundle of 58 residues providing an interface on two of its alpha-helices (Nygren); engineered Kunitz domains based on a small (ca. 58 residues) and robust, disulphide-crosslinked serine protease inhibitor, typically of human origin (e.g. LACI-D1), which can be engineered for different protease specificities (Nixon and Wood); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra 2008); DARPins, designed ankyrin repeat domains (166 residues), which provide a rigid interface arising from typically three repeated beta-turns (Stumpp et al.); avimers (multimerized LDLR-A module) (Silverman et al.); and cysteine-rich knottin peptides (Kolmar).

The term “aptamer” refers to single-stranded or double-stranded oligo-DNA, oligo-RNA or oligo-DNA/RNA or any analogue thereof that specifically binds to a target molecule such as a peptide. Advantageously, aptamers display fairly high specificity and affinity (e.g., K_{A} in the order 1×10⁹ M⁻¹) for their targets. Aptamer production is described inter alia in US 5,270,163; Ellington & Szostak 1990 (Nature 346: 818-822); Tuerk & Gold 1990 (Science 249: 505-510); or “The Aptamer Handbook: Functional Oligonucleotides and Their Applications”, by Klussmann, ed., Wiley-VCH 2006, ISBN 3527310592, incorporated by reference herein.

The term “photoaptamer” refers to an aptamer that contains one or more photoreactive functional groups that can covalently bind to or crosslink with a target molecule.

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term “peptidomimetic” refers to a non-peptide agent that is a topological analogue of a corresponding peptide. Methods of rationally designing peptidomimetics of peptides are known in the art. For example, the rational design of three peptidomimetics based on the sulphated 8-mer peptide CCK26-33, and of two peptidomimetics based on the 11-mer peptide Substance P, and related peptidomimetic design principles, are described in Horwell 1995 (Trends Biotechnol 13: 132-134).

The term “gene-editing system” or “genome editing system” as used herein refers to a tool to induce one or more nucleic acid modifications, such as DNA or RNA modifications, into a specific DNA or RNA sequence within a cell. Targeted genome modification is a powerful tool for genetic manipulation of cells and organisms, including mammals. Genome modification or gene editing, including insertion, deletion or replacement of DNA in the genome, can be carried out using a variety of known gene editing systems. Gene editing systems typically make use of an agent capable of inducing a nucleic acid modification. In certain embodiments, the agent capable of inducing a nucleic acid modification may be a (endo)nuclease or a variant thereof having altered or modified activity. (endo)Nucleases typically comprise programmable, sequence-specific DNA- or RNA-binding modules linked to a nonspecific DNA or RNA cleavage domain. In DNA, these nucleases create site-specific double-strand breaks at desired locations in the genome. The induced double-stranded breaks are repaired through nonhomologous end-joining or homologous recombination, resulting in targeted mutations. In certain embodiments, said (endo)nuclease may be RNA-guided. In certain embodiments, said (endo)nuclease can be engineered nuclease such as a Clustered Regularly Interspaced Short Palindromic Repeats (CRISPR) associated (Cas) (endo)nuclease, such as Cas9, Cpf1, or C2c2, a (zinc finger nuclease (ZFN),a transcription factor-like effector nuclease (TALEN), a meganuclease, or modifications thereof. Methods for using TALEN technology, Zinc Finger technology and CRISPR/Cas technology are known by the skilled person.

The term “nucleic acid” as used herein encompasses DNA, RNA and DNA/RNA hybrid molecules, specifically including hnRNA, pre-mRNA, mRNA, cDNA, genomic DNA, amplification products, oligonucleotides, and synthetic (e.g., chemically synthesised) DNA, RNA or DNA/RNA hybrids. RNA is inclusive of RNAi (inhibitory RNA), dsRNA (double stranded RNA), siRNA (small interfering RNA), mRNA (messenger RNA), miRNA (micro-RNA), tRNA (transfer RNA, whether charged or discharged with a corresponding acylated amino acid), and cRNA (complementary RNA). A nucleic acid can be naturally occurring, e.g., present in or isolated from nature, can be recombinant, i.e., produced by recombinant DNA technology, and/or can be, partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear.

The term “oligonucleotide” as used throughout this specification refers to a nucleic acid (including nucleic acid analogues and mimetics) oligomer or polymer as defined herein. Preferably, an oligonucleotide, such as more particularly an antisense oligonucleotide, is (substantially) single-stranded. Oligonucleotides as intended herein may be preferably between about 10 and about 100 nucleoside units (i.e., nucleotides or nucleotide analogues) in length, preferably between about 15 and about 50, more preferably between about 20 and about 40, also preferably between about 20 and about 30. Oligonucleotides as intended herein may comprise one or more or all non-naturally occurring heterocyclic bases and/or one or more or all non-naturally occurring sugar groups and/or one or more or all non-naturally occurring inter-nucleoside linkages, the inclusion of which may improve properties such as, for example, increased stability in the presence of nucleases and increased hybridization affinity, increased tolerance for mismatches, etc.

Nucleic acid binding agents, such as oligonucleotide binding agents, are typically at least partly antisense to a target nucleic acid of interest. The term “antisense” generally refers to an agent (e.g., an oligonucleotide) configured to specifically anneal with (hybridize to) a given sequence in a target nucleic acid, such as for example in a target DNA, hnRNA, pre-mRNA or mRNA, and typically comprises, consist essentially of or consist of a nucleic acid sequence that is complementary or substantially complementary to said target nucleic acid sequence. Antisense agents suitable for use herein, such as hybridization probes or amplification or sequencing primers and primer pairs) may typically be capable of annealing with (hybridizing to) the respective target nucleic acid sequences at high stringency conditions, and capable of hybridizing specifically to the target under physiological conditions. The terms “complementary” or “complementarity” as used throughout this specification with reference to nucleic acids, refer to the normal binding of single-stranded nucleic acids under permissive salt (ionic strength) and temperature conditions by base pairing, preferably Watson-Crick base pairing. By means of example, complementary Watson-Crick base pairing occurs between the bases A and T, A and U or G and C. For example, the sequence 5′-A-G-U-3′ is complementary to sequence 5′-A-C-U-3′.

The reference to oligonucleotides may in particular but without limitation include hybridization probes and/or amplification primers and/or sequencing primers, etc., as commonly used in nucleic acid detection technologies.

In embodiments of the uses or methods as taught herein, the therapeutic agent may be comprised in a delivery vehicle such as in a viral vector or in a nanoparticle. In embodiments of the uses or methods as taught herein, the therapeutic agent and the dye may be comprised in a nanoparticle.

In embodiments of the uses or methods as taught herein, the therapeutic agent may be a retinal disease drug or a choroidal disease drug.

Retinal or choroidal disease drugs include proteins such as avacincaptad pegol.

Retinal or choroidal disease drugs include antibodies such as bevacizumab, brolucizumab, pegatanib (Macugen^{®}), ranibizumab (Lucentis^{®}), aflibercept (Eylea^{®}).

In embodiments of the uses or methods as taught herein, the therapeutic agent may have a size of at least 5 nm. In embodiments, the therapeutic agent may have a size of at least 10 nm, at least 20 nm, at least 30 nm, at least 40 nm, at least 50 nm, at least 60 nm, at least 70 nm, at least 80 nm, at least 90 nm, or at least 100 nm. In embodiments, the therapeutic agent or the delivery vehicle may have a size of at least 150 nm, at least 200 nm, at least 300 nm, at least 400 nm or at least 500 nm. The present method may enhance the efficacy of delivery of therapeutic agents or their delivery vehicles to the retina or choroid through the perforations in the ILM.

In embodiments of the uses or methods as taught herein, the therapeutic agent or the delivery vehicle may have a size of about 5 nm to about 500 nm. In embodiments, the therapeutic agent or the delivery vehicle may have a size of about 10 nm to about 400 nm, of about 50 nm to about 300 nm, or of about 100 nm to about 200 nm. Such therapeutic agents or delivery vehicles may advantageously be delivered to the retina or the choroid through the perforations in the ILM.

In embodiments, the method comprises administering the therapeutic agent to the vitreous chamber of an eye of the subject. In embodiments of the uses and methods as taught herein, the therapeutic agent may be administered to the vitreous chamber by injection.

In embodiments, the dye and/or the therapeutic agent may be administered to the vitreous chamber comprising the vitreous body. Hence, in embodiments, the dye and/or the therapeutic agent may be administered to the vitreous body of the eye of the subject. In embodiments of the uses and methods as taught herein, the dye and/or the therapeutic agent may be administered to the vitreous chamber by intravitreal administration. Intravitreal administration advantageously allows delivery of the dye and/or the therapeutic agent directly to the vitreous body. In embodiments of the uses or methods as taught herein, the dye and/or the therapeutic agent may be administered to the vitreous body by intravitreal injection. Intravitreal injection allows delivery of the dye and/or the therapeutic agent directly to the vitreous body by a minimally invasive technique, thereby reducing the risks and pain for the patient and increasing the patient’s well-being.

In embodiments, the dye and/or the therapeutic agent may be administered to the vitreous chamber after removal of the vitreous body (i.e. after vitrectomy). Thus, in embodiments, the dye and/or the therapeutic agent may be administered to the vitreous chamber no longer comprising the vitreous body. In embodiments of the uses or methods as taught herein, the dye and/or the therapeutic agent may be administered by injection into the vitreous chamber after removal of the vitreous body.

After vitrectomy, the vitreous may be briefly exchanged by air, after which the dye and/or the therapeutic agent can be flushed through the eye followed by filling the eye with the replacement (i.e. substitute) of the vitreous, such as a BSS, gas (e.g. perfluoropropane), or silicone oil. Or after replacement of the vitreous, the dye and/or the therapeutic agent can be administered into the replacement (i.e. substitute) of the vitreous, such as a BSS, gas (e.g. perfluoropropane), or silicone oil. In embodiments, the dye and/or the therapeutic agent may be injected into the replacement of the vitreous body, e.g. into the BSS, gas, or silicone oil, after removal of the vitreous body.

In embodiments, the treatment of a retinal or choroidal disease may comprise performing laser irradiation of the dye as taught herein, in particular pulsed laser irradiation of the dye as taught herein. Accordingly, in embodiments, the treatment as taught herein comprises laser-assisted treatment. The terms “laser-assisted treatment” or “phototherapy” may be used interchangeably herein.

Further aspects relate to:
a dye and a therapeutic agent for use in a method of laser-assisted treatment of a retinal disease or a choroidal disease (of an eye) in a subject.
a method of laser-assisted treatment of a retinal disease or a choroidal disease in a subject in need of such a treatment, comprising administering a therapeutically effective amount of a dye and a therapeutic agent to the subject.
the use of a dye and a therapeutic agent for the manufacture of a medicament for laser-assisted treatment of a retinal disease or a choroidal disease in a subject.
the use of a dye and a therapeutic agent for laser-assisted treatment of a retinal disease or a choroidal disease in a subject.

The dye as taught herein and the therapeutic agent as taught herein allow treatment, such as laser-assisted treatment, of a retinal disease or a choroidal disease.

As used herein, a phrase such as “a subject in need of treatment” includes subjects that would benefit from treatment of a given condition, particularly a retinal disease or a choroidal disease. Such subjects may include, without limitation, those that have been diagnosed with said condition, those prone to develop said condition and/or those in who said condition is to be prevented.

The terms “treat” or “treatment” encompass both the therapeutic treatment of an already developed disease or condition, such as the therapy of an already developed retinal disease or choroidal disease, as well as prophylactic or preventive measures, wherein the aim is to prevent or lessen the chances of incidence of an undesired affliction, such as to prevent occurrence, development and progression of a retinal or choroidal disease. Beneficial or desired clinical results may include, without limitation, alleviation of one or more symptoms or one or more biological markers, diminishment of extent of disease, stabilized (i.e., not worsening) state of disease, delay or slowing of disease progression, amelioration of the disease state, and the like. The term may encompass ex vivo or in vivo treatments.

The uses and methods as taught herein allow to administer a therapeutically effective amount of the active compound, such as the dye as taught herein and/or the therapeutic agent as taught herein, in subjects having a retinal or choroidal disease which will benefit from such treatment. The term “therapeutically effective amount” as used herein, refers to an amount of active compound that elicits the biological or medicinal response in a subject that is being sought by a surgeon, researcher, veterinarian, medical doctor or other clinician, which may include inter alia alleviation of the symptoms of the disease or condition being treated.

The term “therapeutically effective dose” refers to an amount of an active compound, such as the dye as taught herein and/or the therapeutic agent as taught herein, that when administered brings about a positive therapeutic response with respect to treatment of a patient having the disease or condition being treated, such as a retinal disease or choroidal disease.

Appropriate therapeutically effective doses of an active compound, such as the dye as taught herein and/or the therapeutic agent as taught herein, may be determined by a qualified physician with due regard to the nature of the agent, the disease condition and severity, and the age, size and condition of the patient.

In certain embodiments, the active compound, such as the dye as taught herein and/or the therapeutic agent as taught herein, may be formulated into and administered as pharmaceutical formulations or pharmaceutical compositions.

In certain embodiments, the active compound, such as the dye as taught herein and/or the therapeutic agent as taught herein, may be formulated into a kit of parts and administered simultaneously or separately.

In embodiments, the dye may be comprised in a pharmaceutical formulation. In embodiments, the therapeutic agent may be comprised in a pharmaceutical formulation.

In embodiments, the dye and the therapeutic agent may be comprised in a pharmaceutical formulation.

The dye or pharmaceutically acceptable salts thereof, and/or the therapeutic agent or pharmaceutically acceptable salts thereof can be formulated as an aqueous solution.

Accordingly, an aspect relates to a pharmaceutical formulation comprising a dye as taught herein. A further aspect provides a pharmaceutical formulation comprising a dye as taught herein and a therapeutic agent as taught herein.

A further aspect relates to a pharmaceutical formulation as taught herein for use in a method of treating a retinal disease or a choroidal disease (of an eye) in a subject. Preferably, the subject is a human subject.

The terms “pharmaceutical composition”, “pharmaceutical formulation” or “pharmaceutical preparation” may be used interchangeably herein and refer to a mixture comprising an active ingredient. The terms “composition” or “formulation” may likewise be used interchangeably herein.

The terms “active ingredient”, “active compound” or “active component” can be used interchangeably and broadly refer to a compound or substance which, when provided in an effective amount, achieves a desired outcome. The desired outcome may be therapeutic and/or prophylactic. Typically, an active ingredient may achieve such outcome(s) through interacting with and/or modulating living cells or organisms.

The term “active” in the recitations “active ingredient” or “active component” refers to “pharmacologically active” and/or “physically active”.

In embodiments, the pharmaceutical formulations as taught herein may comprise in addition to the dye and the therapeutic agent one or more pharmaceutically acceptable excipients.

The term “pharmaceutically acceptable” as used herein is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, antioxidants, tonicity controlling agents, absorption delaying agents, and the like. The use of such media and agents for pharmaceutical active substances is well known in the art. Except insofar as any conventional media or agent is incompatible with the active substance, its use in the therapeutic compositions may be contemplated.

Pharmaceutical compositions as intended herein may be formulated for essentially any route of administration, such as without limitation, oral administration (such as, e.g., oral ingestion), parenteral administration (such as, e.g., subcutaneous, intravenous or intramuscular injection or infusion), and the like.

For example, for oral administration, pharmaceutical compositions may be formulated in the form of pills, tablets, lacquered tablets, coated (e.g., sugar-coated) tablets, granules, hard and soft gelatin capsules, aqueous, alcoholic or oily solutions, syrups, emulsions or suspensions. In an example, without limitation, preparation of oral dosage forms may be is suitably accomplished by uniformly and intimately blending together a suitable amount of the active compound in the form of a powder, optionally also including finely divided one or more solid carrier, and formulating the blend in a pill, tablet or a capsule. Exemplary but non-limiting solid carriers include calcium phosphate, magnesium stearate, talc, sugars (such as, e.g., glucose, mannose, lactose or sucrose), sugar alcohols (such as, e.g., mannitol), dextrin, starch, gelatin, cellulose, polyvinylpyrrolidine, low melting waxes and ion exchange resins. Compressed tablets containing the pharmaceutical composition can be prepared by uniformly and intimately mixing the active ingredient with a solid carrier such as described above to provide a mixture having the necessary compression properties, and then compacting the mixture in a suitable machine to the shape and size desired. Moulded tablets maybe made by moulding in a suitable machine, a mixture of powdered compound moistened with an inert liquid diluent. Suitable carriers for soft gelatin capsules and suppositories are, for example, fats, waxes, semisolid and liquid polyols, natural or hardened oils, etc.

Preferably the pharmaceutical formulation may be formulated for parenteral administration such as administration into the vitreous chamber, in particular intravitreal administration or administration into a replacement of the vitreous body, e.g. by injection.

In embodiments, the pharmaceutical composition may be formulated as an aqueous solution. For example, for parenteral administration, pharmaceutical compositions may be advantageously formulated as solutions, suspensions or emulsions with suitable solvents, diluents, solubilisers or emulsifiers, etc. Suitable solvents are, without limitation, water, physiological saline solution or alcohols, e.g. ethanol, propanol, glycerol, in addition also sugar solutions such as glucose, invert sugar, sucrose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer’s solution or isotonic sodium chloride solution, or suitable dispersing or wetting and suspending agents, such as sterile, bland, fixed oils, including synthetic mono- or diglycerides, and fatty acids, including oleic acid. The dye or pharmaceutically acceptable salts thereof, and/or the therapeutic agent or pharmaceutically acceptable salts thereof can also be lyophilized. The obtained lyophilizates can be used, for example, for injection or infusion preparation or for the production of injection or infusion preparations.

In embodiments, the dye may be comprised in a kit of parts.

In embodiments, the dye and the therapeutic agent may be comprised in a kit of parts.

A further aspect relates to a kit of parts comprising a dye as taught herein. A further aspect provides a kit of parts comprising a dye as taught herein and a therapeutic agent as taught herein.

A further aspect thus relates to a kit of parts as taught herein for use in a method of treating a retinal disease or choroidal disease (of an eye) in a subject. Preferably, the subject is a human subject.

The terms “kit of parts”, “kit-of-parts” or “kit” as used herein refer to a product containing components necessary for carrying out the specified uses or methods, packed so as to allow their transport and storage. Materials suitable for packing the components comprised in a kit include crystal, plastic (e.g., polyethylene, polypropylene, polycarbonate), bottles, flasks, vials, ampules, paper, envelopes, or other types of containers, carriers or supports. Where a kit comprises a plurality of components, at least a subset of the components (e.g., two or more of the plurality of components) or all of the components may be physically separated, e.g., comprised in or on separate containers, carriers or supports. The components comprised in a kit may be sufficient or may not be sufficient for carrying out the specified uses or methods, such that external reagents or substances may not be necessary or may be necessary for performing the methods, respectively. Typically, kits are employed in conjunction with standard laboratory equipment, such as liquid handling equipment, environment (e.g., temperature) controlling equipment, analytical instruments, etc. In addition to the dye as taught herein and/or the therapeutic agent as taught herein, optionally provided on arrays or microarrays, the present kits may also include excipients such as solvents useful in the specified uses or methods. Typically, the kits may also include instructions for use thereof, such as on a printed insert or on a computer readable medium. The terms may be used interchangeably with the term “article of manufacture”, which broadly encompasses any man-made tangible structural product, when used in the present context.

A further aspect provides a method for delivering a therapeutic agent to the retina or the choroid of an eye in a subject, the method comprising:
administering a dye to the vitreous chamber of the eye of the subject;
irradiating at least part of the ILM of the eye of the subject, thereby photoporating at least part of the ILM; and
administering the therapeutic agent to the vitreous chamber of the eye of the subject;
or the method comprising:
administering the dye and the therapeutic agent to the vitreous chamber of an eye of the subject; and
irradiating at least part of the ILM of the eye of the subject, thereby photoporating at least part of the ILM.

A related aspect provides a dye for use in a method of delivering a therapeutic agent to the retina or the choroid of an eye in a subject, the method comprising:
administering the dye to the vitreous chamber of the eye of the subject;
irradiating at least part of the ILM of the eye of the subject, thereby photoporating at least part of the ILM; and
administering the therapeutic agent to the vitreous chamber of the eye of the subject;
or the method comprising:
administering the dye and the therapeutic agent to the vitreous chamber of an eye of the subject; and
irradiating at least part of the ILM of the eye of the subject, thereby photoporating at least part of the ILM.

The present application also provides aspects and embodiments as set forth in the following Statements:

Statement 1. A dye for use in a method of photoporation of the inner limiting membrane (ILM) of an eye in a subject.

Statement 2. The dye for use according to statement 1, wherein the method comprises:
administering the dye to the vitreous chamber of the eye of the subject; and
irradiating at least part of the ILM of the eye of the subject, thereby photoporating at least part of the ILM in the subject.

Statement 3. A dye and a therapeutic agent for use in a method of treating a retinal disease or a choroidal disease of an eye in a subject.

Statement 4. The dye and therapeutic agent for use according to statement 3, wherein the method comprises:
administering the dye to the vitreous chamber of the eye of the subject;
irradiating at least part of the ILM of the eye of the subject, thereby photoporating at least part of the ILM; and
administering the therapeutic agent to the vitreous chamber of the eye of the subject, thereby treating the retinal disease or the choroidal disease in the subject;
or wherein the method comprises:
administering the dye and the therapeutic agent to the vitreous chamber of an eye of the subject; and
irradiating at least part of the ILM of the eye of the subject, thereby photoporating at least part of the ILM and treating the retinal disease or the choroidal disease in the subject.

Statement 5. The dye for use according to statement 1 or 2, or the dye and therapeutic agent for use according to claim 3 or 4, wherein the dye is a vital dye.

Statement 6. The dye for use according to any one of statements 1, 2, or 5, or the dye and therapeutic agent for use according to any one of statements 3 to 5, wherein the dye is a vital dye selected from the group consisting of: Indocyanine Green (ICG), Trypan Blue (TB), Brilliant Blue (BB), Janus green B (JG), Gentian violet (GV), Bromophenol Blue (BPB), Patent blue (PB), Light Green (LG), Fast Green (FG), Infracyanine Green (IfCG), Methylene blue (MB), Toluidine blue (ToB), Fluorescein Sodium (FS), Rose Bengal (RB), and Rhodamine 6G (R6G); preferably wherein the dye is ICG.

Statement 7. The dye for use according to any one of statements 1, 2, 5 or 6, or the dye and therapeutic agent for use according to any one of statements 3 to 6, wherein the dye is administered at a concentration of about 0.001 mg/ml to about 0.5 mg/ml.

Statement 8. The dye for use according to any one of statements 1, 2, or 5 to 7, or the dye and therapeutic agent for use according to any one of statements 3 to 7, wherein the dye is conjugated to a further agent; wherein the dye is comprised in a nanoparticle; and/or wherein the dye and the therapeutic agent are comprised in a nanoparticle.

Statement 9. The dye and therapeutic agent according to any one of statements 3 to 8, wherein the therapeutic agent is a retinal disease drug or a choroidal disease drug.

Statement 10. The dye and therapeutic agent according to any one of statements 3 to 9, wherein the therapeutic agent is selected from the group consisting of a stem cell, a protein, a polypeptide, a peptide, an antibody, an antibody fragment, an antibody-like protein scaffold, an aptamer, a photoaptamer, a spiegelmer, a peptidomimetic, a gene-editing system, a nucleic acid, and combinations thereof.

Statement 11. The dye and therapeutic agent according to any one of statements 3 to 10, wherein the therapeutic agent is comprised in a viral vector or nanoparticle.

Statement 12. The dye for use according to any one of statements 1, 2, or 5 to 8, or the dye and therapeutic agent for use according to any one of claims 3 to 11, wherein the dye and/or the therapeutic agent is administered to the vitreous body by intravitreal injection.

Statement 13. The dye for use according to any one of statements 1, 2, 5 to 8, or 12, or the dye and therapeutic agent for use according to any one of statements 3 to 12, wherein the dye and/or the therapeutic agent is administered by injection into the vitreous chamber after removal of the vitreous body.

Statement 14. The dye for use according to any one of statements 1, 2, 5 to 8, or 12 to 13, or the dye and therapeutic agent for use according to any one of statements 3 to 13, wherein the at least part of the ILM is irradiated with electromagnetic radiation; preferably wherein the at least part of the ILM is irradiated with laser radiation; more preferably wherein the at least part of the ILM is irradiated with pulsed-laser radiation.

Statement 15. A method for delivering a therapeutic agent to the retina or the choroid of an eye in a subject, the method comprising:
administering a dye to the vitreous chamber of the eye of the subject;
irradiating at least part of the ILM of the eye of the subject, thereby photoporating at least part of the ILM; and
administering the therapeutic agent to the vitreous chamber of the eye of the subject;
or the method comprising:
administering the dye and the therapeutic agent to the vitreous chamber of an eye of the subject; and
irradiating at least part of the ILM of the eye of the subject, thereby photoporating at least part of the ILM.

Statement 16. A vital dye for use in a method of photoporation of the ILM of an eye in a subject.

Statement 17. A method of photoporation of the ILM of an eye in a subject in need of such a treatment, comprising administering a therapeutically effective amount of a vital dye to the subject.

Statement 18. Use of a vital dye for the manufacture of a medicament for the photoporation of the ILM of an eye in a subject.

Statement 19. Use of a vital dye for photoporation of the ILM of an eye of a subject.

Statement 20. A vital dye for use in a method of treatment of a retinal disease or a choroidal disease in a subject.

Statement 21. A method of treatment of a retinal disease or a choroidal disease in a subject in need of such a treatment, comprising administering a therapeutically effective amount of a vital dye to the subject.

Statement 22. Use of a vital dye for the manufacture of a medicament for the treatment of a retinal disease or a choroidal disease.

Statement 23. Use of a vital dye for the treatment of a retinal disease or a choroidal disease.

Statement 24. The vital dye for use according to any one of statements 16 to 23, wherein the method comprises:
administering the vital dye to the vitreous chamber of the eye of the subject; and
irradiating at least part of the ILM of the eye of the subject, thereby photoporating at least part of the ILM in the subject.

Statement 25. A vital dye and a therapeutic agent for use in a method of treatment of a retinal disease or a choroidal disease of an eye in a subject.

Statement 26. A method of treatment of a retinal disease or a choroidal disease in a subject in need of such a treatment, comprising administering a therapeutically effective amount of a vital dye and a therapeutic agent to the subject.

Statement 27. Use of a vital dye and a therapeutic agent for the manufacture of a medicament for the treatment of a retinal disease or a choroidal disease.

Statement 28. Use of a vital dye and a therapeutic agent for the treatment of a retinal disease or a choroidal disease.

Statement 29. The vital dye and therapeutic agent for use according to any one of statements 25 to 28, wherein the method comprises:
administering the vital dye to the vitreous chamber of the eye of the subject;
irradiating at least part of the ILM of the eye of the subject, thereby photoporating at least part of the ILM; and
administering the therapeutic agent to the vitreous chamber of the eye of the subject, thereby treating the retinal disease or the choroidal disease in the subject;
or wherein the method comprises:
administering the vital dye and the therapeutic agent to the vitreous chamber of an eye of the subject; and
irradiating at least part of the ILM of the eye of the subject, thereby photoporating at least part of the ILM and treating the retinal disease or the choroidal disease in the subject.

Statement 30. The vital dye and therapeutic agent according to any one of statements 25 to 29, wherein the therapeutic agent is a retinal disease drug or a choroidal disease drug.

Statement 31. The vital dye and therapeutic agent according to any one of statements 25 to 30, wherein the therapeutic agent is selected from the group consisting of a stem cell, a protein, a polypeptide, a peptide, an antibody, an antibody fragment, an antibody-like protein scaffold, an aptamer, a photoaptamer, a spiegelmer, a peptidomimetic, a gene-editing system, a nucleic acid, and combinations thereof.

Statement 32. The vital dye and therapeutic agent according to any one of statements 25 to 31, wherein the therapeutic agent is comprised in a viral vector or in a nanoparticle.

Statement 33. The vital dye for use according to any one of statements 16 to 24, or the vital dye and therapeutic agent for use according to any one of statements 25 to 32, wherein the vital dye is selected from the group consisting of: Indocyanine Green (ICG), Trypan Blue (TB), Brilliant Blue (BB), Janus green B (JG), Gentian violet (GV), Bromophenol Blue (BPB), Patent blue (PB), Light Green (LG), Fast Green (FG), Infracyanine Green (IfCG), Methylene blue (MB), Toluidine blue (ToB), Fluorescein Sodium (FS), Rose Bengal (RB), and Rhodamine 6G (R6G).

Statement 34. The vital dye for use according to any one of statements 16 to 24, or 33, or the vital dye and therapeutic agent for use according to any one of statements 25 to 33, wherein the vital dye is administered at a concentration of about 0.001 mg/ml to about 5 mg/ml or of about 0.001 mg/ml to about 0.5 mg/ml.

Statement 35. The vital dye for use according to any one of statements 16 to 24, 33 or 34, or the vital dye and therapeutic agent for use according to any one of statements 25 to 34, wherein the vital dye is conjugated to a further agent; wherein the vital dye is comprised in a nanoparticle; and/or wherein the vital dye and the therapeutic agent are comprised in a nanoparticle.

Statement 36. The vital dye for use according to any one of statements 16 to 24, or 33 to 35, or the vital dye and therapeutic agent for use according to any one of statements 25 to 35, wherein the vital dye and/or the therapeutic agent is administered to the vitreous body by intravitreal injection.

Statement 37. The vital dye for use according to any one of statements 16 to 24, or 33 to 36, or the vital dye and therapeutic agent for use according to any one of statements 25 to 36, wherein the vital dye and/or the therapeutic agent is administered by injection into the vitreous chamber after removal of the vitreous body.

Statement 38. The vital dye for use according to any one of statements 16 to 24, or 33 to 37, or the vital dye and therapeutic agent for use according to any one of statements 25 to 37, wherein the at least part of the ILM is irradiated with electromagnetic radiation; preferably wherein the at least part of the ILM is irradiated with laser radiation; more preferably wherein the at least part of the ILM is irradiated with pulsed-laser radiation.

Statement 39. A method for delivering a therapeutic agent to the retina or the choroid of an eye in a subject, the method comprising:
administering a vital dye to the vitreous chamber of the eye of the subject;
irradiating at least part of the ILM of the eye of the subject, thereby photoporating at least part of the ILM; and
administering the therapeutic agent to the vitreous chamber of the eye of the subject;
or the method comprising:
administering the vital dye and the therapeutic agent to the vitreous chamber of an eye of the subject; and
irradiating at least part of the ILM of the eye of the subject, thereby photoporating at least part of the ILM.

The above aspects and embodiments are further supported by the following non-limiting examples.

### EXAMPLES

### Example 1: ILM Perforation by Photoporation Using Free Indocyanine Green According to Embodiments of the Invention

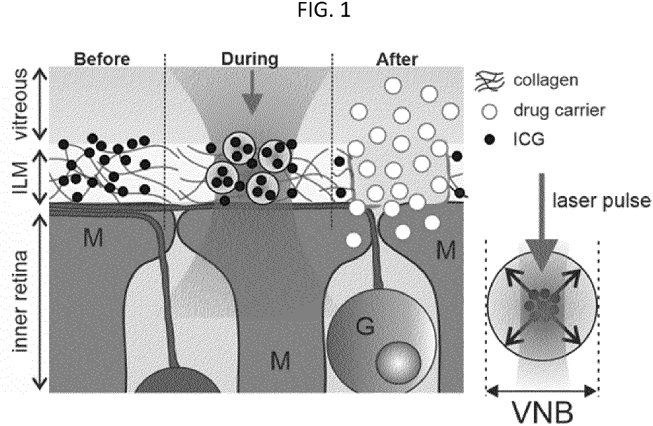

The study aimed to perforate the ILM by photoporation. Thereto, a vital dye, in particular Indocyanine Green (ICG), was delivered to the ILM after which the retina was illuminated with a powerful but extremely short pulse of 800 nm laser light. Without being bound to theory, the absorbance of light by the ICG can result in the evaporation of the surrounding water, creating vapour nanobubbles (VNBs) which upon collapse can cause damage to neighbouring tissue such as in particular the ILM. An overview of the photoporation concept is shown in FIG. 1.

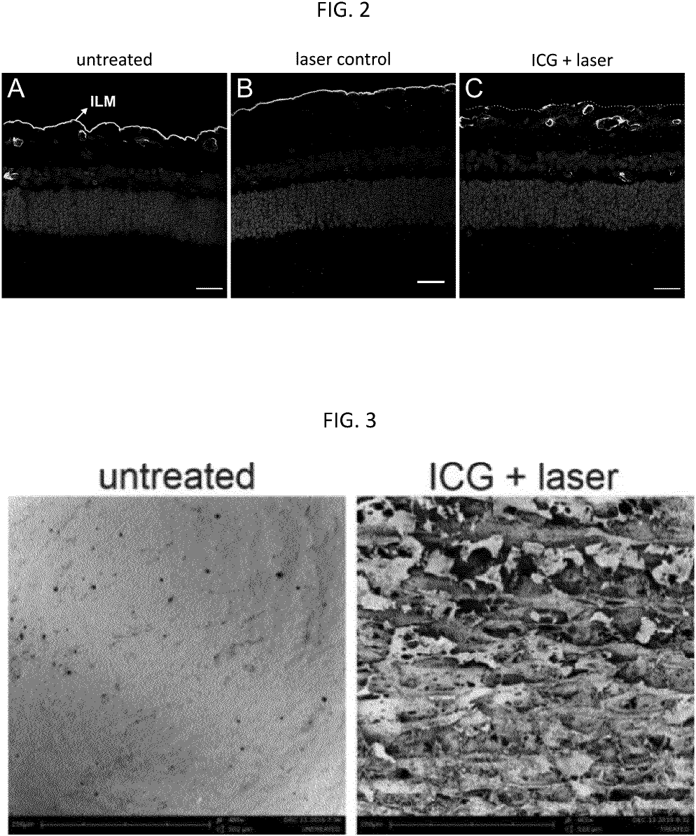

To generate ILM perforation, an ICG solution (1 mg/ml) was applied on the ILM surface of bovine retinal explants followed by laser scanning of the tissue with 2 picosecond pulses. As shown in FIGS. 2A and B, the ILM (indicated by dashed white line) of untreated retinal explants and laser-only treated explants was intact. In explants treated with both ICG and laser light, however, most of the ILM was absent with only small parts remaining (FIG. 2C, dashed with line).

Subsequently, the application of several complementary techniques was tested in order to prove ILM ablation as witnessed on cryosections. A first technique was Scanning Electron Microscopy (SEM). Thereto, bovine retinal explants were incubated with ICG followed by laser treatment. After freeze-drying and sample preparation, SEM images of the retinal surface were taken. As shown in FIG. 3, the retinal surface of untreated explants had a smooth appearance with few small holes (likely due to sample preparation). In contrast, the retinal surface of treated explants was severely disorganized and damaged, proving the power of our ICG-mediated photoporation.

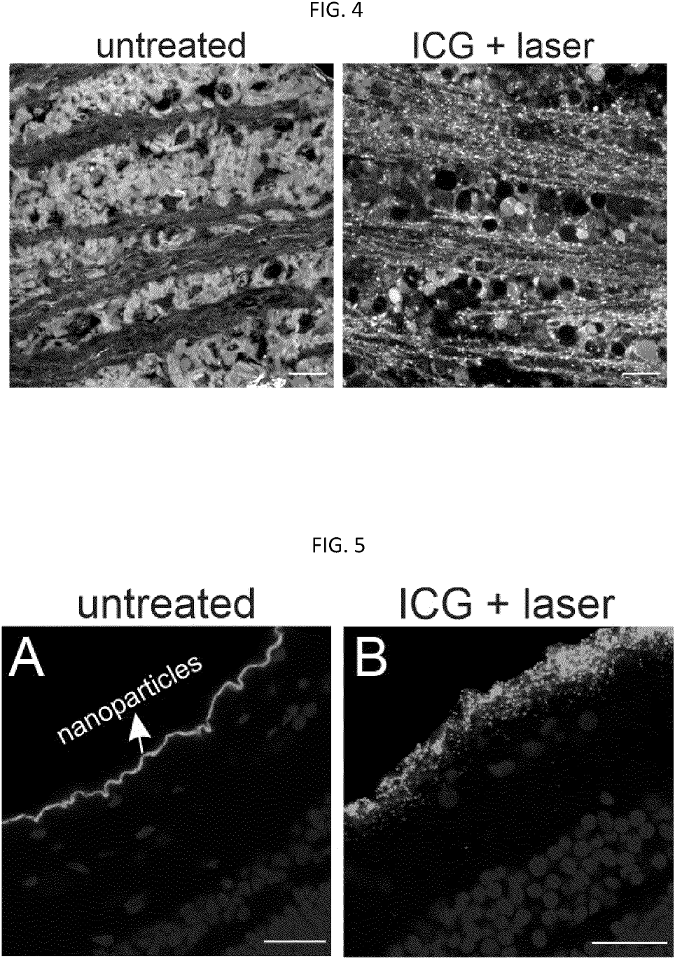

Furthermore, a method was tested to look at the retinal surface in more detail, allowing to visualize several types of structures such as nerve fibers, blood vessels, and Müller cells (cell type located right underneath the ILM). To this end, intact retinal explants were stained with a dye mixture of Mitotracker Deep Red and Hoechst, which allows to identify these structures. FIG. 4 shows that the trend witnessed with cryosection staining and SEM (FIG. 3) was confirmed applying this method. Indeed, in untreated explants, the retinal surface was neatly organized: a mosaic of Müller cell endfeet intertwined with bundles of nerve fibers. Explants treated with ICG and laser showed a similar pattern but way more disorganized: the nerve bundles had a more granular appearance and the neat mosaic of Müller cell endfeet was ruptured (FIG. 4).

It was further tested whether ICG-mediated ILM ablation allowed to deliver therapeutics to the retina. It was found that ICG-mediated ILM perforation significantly enhanced the retinal entry of 100 nm sized polystyrene nanoparticles (Fluospheres) used as a model to validate the delivery of a therapeutic agent. As shown in FIG. 5A, 100 nm Fluospheres were not able of crossing the intact ILM in untreated bovine explants, again demonstrating the barrier role of the ILM. After destruction of the ILM by photoporation, however, massive entry of the nanoparticles into the retina was observed (FIG. 5B).

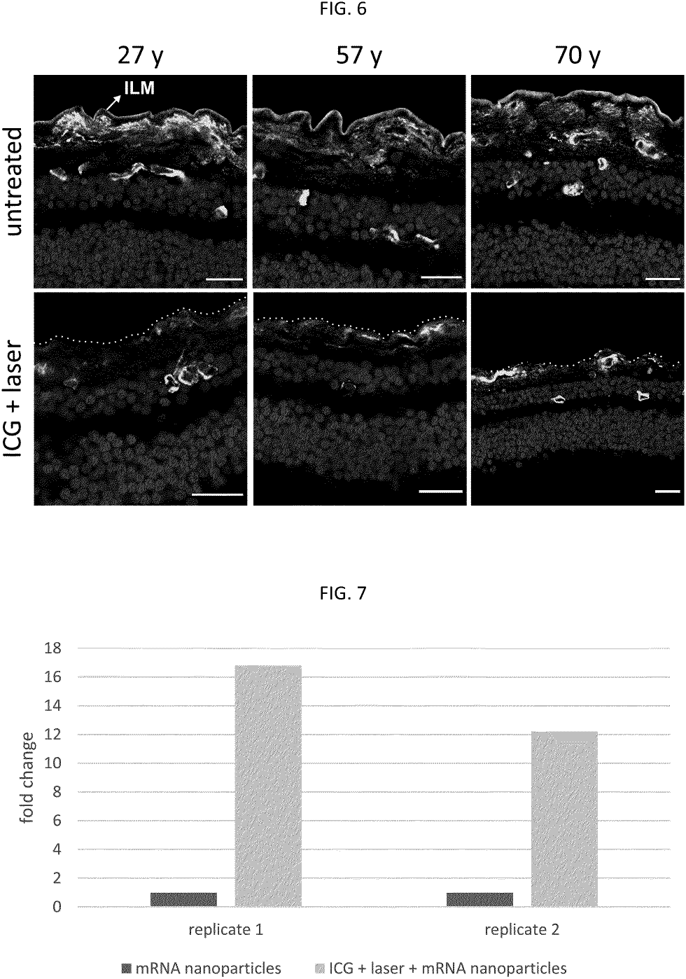

It is well-established that the human retina exhibits a thicker and more complex ILM in comparison to all animal models. Therefore, the approach was tested on human samples. Since the thickness of the ILM is also age dependent, we investigated human ILM ablation in younger and older patients. FIG. 6 shows images of cryosections of human explants stained for nuclei in the retina by Hoechst staining (indicated in grey - low contrast) and showing the ILM stained by antibodies against Collagen IV (indicated in white). As illustrated in the top row, the ILM in untreated explants becomes gradually thicker with increasing age, which is an observation in accordance with literature (FIG. 6, untreated). Treated samples (bottom row) were treated with 1 mg/ml ICG and 0,4 J/cm2 laser fluence. Absence of the ILM is indicated by a dashed line. In case of the 27 year old and 57 year old patient the ILM was completely absent, while the treated retina of the 70 year old patient exhibited a fragmented ILM (FIG. 6, ICG + laser).

We further investigated retinal mRNA expression of luciferase mRNA after 24 h retinal culture in bovine explants treated with mRNA nanoparticles (dark grey) and bovine explants treated with ICG, laser and mRNA nanoparticles (light grey). As shown in the FIG. 7, the photoporation approach illustrating the invention resulted in a 12- to 17-fold increase in mRNA expression in the retina compared to explants which did not receive the photoporation treatment.

In brief, Example 1 shows that the photoporation method was able of destroying the ILM using free ICG dye and pulsed laser light. By using this approach, the delivery of nanoparticles into the retina was greatly augmented.

The following examples describe photoporation of the ILM with ICG-loaded nanoparticles (ICG-NPs), with the aim to perforate the ILM and allow better transfer of drug(s) (carriers) from the vitreous into the retina. More specifically, ICG-NPs are synthesized and tested for their VNB-creating capacity and ability to migrate through the vitreoretinal interface (Example 2), after which ICG-NPs incubation and laser treatment is fine-tuned in ex vivo bovine retinal tissue to achieve safe photoporation of the ILM (Example 3). To anticipate on the morphological variability and complexity of the human ILM, known to be highly developed and thick, the treatment is also tested on human retinal tissue in Example 4, where the benefit of the approach towards the delivery of several types of retinal therapeutics (viruses, lipid nanoparticles and antibodies) is also evaluated. Finally, Example 5 describes the in vivo behaviour and toxicity of the ICG-NPs as well as the efficacy and safety of the overall photoporation concept in rabbits.

### Example 2: Synthesis and Characterization of ICG-loaded Nanoparticles According to Embodiments of the Invention

### 1. Synthesis of ICG-Loaded Nanoparticles

Three types of ICG-loaded NPs were selected which meet the following requirements: size larger than 100 nm to prevent ILM passage, neutral to negative charge to facilitate mobility through the vitreous, and high ICG encapsulation efficiency:

Liposomes encapsulating ICG (Lip-ICG): Size: about 100-150 nm; Charge: Negative to neutral (Lajunen et al., 2018, J. Control. Release 284, 213-223).

Human serum albumin ICG nanoparticles (HSA-ICG NPs): Size: about 250 nm; Charge: Negative to neutral (Sheng et al., 2014, ACS Nano, 8(12):12310-22).

Poly(lactic-co-glycolic acid)-based ICG nanoparticles (PLGA-ICG NPs): Size: about 350 nm; Charge: Negative (Saxena et al., 2004, Int J Pharm, 278(2):293-301).

We were capable of reproducing ICG-loaded Human Serum Albumin (HSA) NPs and found them to be efficient in creating VNBs upon laser irradiation (data not shown). ICG-loaded liposomal formulations typically have ICG loaded in the aqueous compartment or incorporated into the lipid membrane by hydrating the lipid film with ICG solution or adding an ICG-methanol mixture to the lipids before evaporation, respectively. To maximize ICG loading within the NP, we optimize the protocols to accomplish both types of encapsulation within one liposome. Following NP synthesis, the size and zeta potential of the NPs is determined with a Nanosizer (Malvern Instruments) in addition to the ICG encapsulation efficiency as measured with a Victor3 plate reader (Perkin Elmer).

### 2. Evaluation of VNB-Creating Ability of ICG-Loaded Nanoparticles

NP-induced VNB formation is detected by dark field microscopy where the VNB size is measured as a function of irradiation parameters. VNB measurements are performed in 1) buffer solution, to assess VNB formation by single NPs, and 2) after centrifugation of the well plate to imitate the accumulation of NPs to a surface like the ILM. In particular, the experiments aim to define suitable conditions to create VNBs of sufficient size to effectively puncture the > 2 µm thick human ILM.

### 3. Affinity of ICG-NPs for Bovine ILM and Their Potential to Migrate Through Vitreous

To investigate that following injection in the vitreous chamber the particles are: 1) able of migrating through the vitreous, and 2) binding to the ILM, two types of studies are carried out as described below.

### 1) ICG-NPs Are Applied on Conventional Retinal Explants

Here, it is examined which NPs have a strong inherent affinity for the ILM without the influence of the vitreous. In absence of the vitreous, the fluorescently labelled ICG-NPs are dripped at on top of conventional retinal explants (w/o vitreous, size about 1 cm²) at varying concentrations and cultured for 24 h. After a washing step, the total load of ICG present in/on the intact explants in function of concentration is quantified with the IVIS imaging system and compared with the data on free ICG incubation. Qualitatively, presence of fluorescently labeled NPs is assessed in retinal cryosections stained for the ILM with anti-Collagen IV antibodies. Importantly, retinal cryosections also allow to assess if NPs are indeed unable to enter the retina.

### 2) ICG-NPs are Injected in Vitreoretinal Explants

Here, fluorescently labelled NPs are injected into the vitreous of a ‘vitreoretinal explant’ (size about 2 cm²), an in-house developed bovine retinal model that keeps a layer of vitreous attached to the retina at all times, ensuring an intact vitreoretinal interface. This model therefore closely resembles the complexity of the in vivo situation and mimics the intended clinical approach. For these experiments, ICG-NPs of varying concentration are administrated by multiple ex vivo intravitreal (IVT) injections (about 50 µl/injection) into the model to ensure NP distribution along the ILM. Since the vitreous represents a diffusional barrier, we look into its influence on distribution and attachment of the NPs to the ILM. After allowing diffusion for 24 hours, the amount of NPs attached to the ILM is checked in retinal cryosections stained for the ILM as well as if NPs do not enter the retina. In addition, we determine the shortest time point (e.g. 4 hours) which provides sufficient NP attachment to the ILM to commence laser treatment.

### Example 3: Efficacy and Safety of ICG-NP Mediated ILM Photoporation in Ex Vivo Bovine Explants

Once the synthesis of VNB-creating NPs is optimized, their potential to perforate the ILM under influence of NIR pulsed laser radiation is evaluated. To this end, bovine vitreoretinal explants are isolated and injected as in Example 2, item 3, 2). Next, the explants are irradiated with laser light such that each location receives exactly one laser pulse of 2 picoseconds. Appropriate controls are taken, including untreated samples, laser-only and NP-only treated samples. All staining described below is imaged with a confocal microscope. The efficacy and safety of ICG-NP mediated ILM perforation is compared to the results obtained with free ICG dye.

### 1. Evaluation of ILM Integrity After ICG-NP Mediated Photoporation

With the optimized laser settings and incubation parameters (Example 2), we irradiate ICG-NP treated explants at various laser fluences after which the integrity of the ILM and the inner retina is evaluated using two methods immediately after laser treatment:
1) retinal cryosections stained for the ILM with anti-Collagen IV antibodies as described above; and
2) retinal surface imaging, where the surface of intact explants is visualized after fixation and incubation with Mitotracker Deep Red (stains Müller cells) and Hoechst.

### 2. Transport of Model Nanoparticles Through the Perforated ILM

Pore formation in the ILM should enhance NP delivery into the retina. To assess this concept, polystyrene (PS) carboxylated NPs (Fluospheres, Molecular Probes) are used since these NPs are highly fluorescent, well characterized, monodisperse and fully mobile in the vitreous. Polystyrene NPs with sizes that are representative for nanoscopic drug carriers are tested: 40 nm is at the upper size limit of viral vectors (about 25 nm), while non-viral carriers have a typical size of 100 to 200 nm. To explore if the formation and collapse of VNB’s is harmful for particles in their close proximity, we will explore two treatment options: 1) PS beads are co-injected with the ICG-NPs into vitreoretinal explants, incubated for a short time point (e.g. 4 hours), laser-treated and cultured overnight; or 2) PS beads are injected after ICG-NP injection and laser treatment, followed by overnight culture. In all cases, cryosection preparation and staining allows to examine the location and extent of penetration of the polystyrene NPs into the retina.

### 3. Influence of ILM Photoporation on Retinal Viability and Morphology

The potential toxicity of the photoporation treatment is assessed ex vivo by cryosection preparation of the vitreoretinal explants followed by staining for the following markers: 1) caspase-3 to detect apoptosis, a common cell death pathway upon light injury; 2) glial fibrillary acidic protein (GFAP), a filamentous protein specific to Müller cells often applied as an early sensitive marker for retinal injury; and/or 3) Hematoxylin and Eosin (H&E) histological staining to provide detailed insight on retinal morphology.

The study allows to optimize ICG-NP incubation and laser treatment for photoporation of the bovine ILM in a safe manner and provides evidence that ICG-NP mediated ILM photoporation succeeds in enhancing drug delivery to the retina.

### Example 4: Perforation of the Human ILM and Enhanced Passage of Therapeutic Carriers Through the Perforated Bovine ILM According to Embodiments of the Invention

### 1. ICG-NP Mediated Photoporation of the Human ILM

A limitation of the bovine retinal explant models is the difference in thickness between the bovine ILM (about 100 —400 nm) and the human ILM (up to 5 µm). In addition, the ILM thickness in humans is known to vary greatly depending on the patient’s age and the region within the eye. Surely, whereas the entire posterior retina is characterized by a thick ILM reaching up to several microns, the foveal pit located at the center of the human macula exhibits an extremely thin ILM (estimated thickness of about 100 nm). Unfortunately, this unique pattern is absent in all large or small laboratory animals (except for non-human primates). With the aim to realistically estimate the clinical potential of our approach, it is tested on human tissue, in particular human ocular rest material, i.e. leftover eyecups from which the corneas are excised for donation. Due to the protocol followed for cornea donation, the retina cannot be used for viability studies and we are only able of isolating conventional retinal explants without vitreous. However, the human retinal samples allow to investigate if the treatment is also able of perforating the more developed human ILM.

### 1) Affinity of ICG-NPs for Bovine Versus Human ILM

In addition to morphology, there is evidence that the composition of the human ILM is very unique among all species, which could have an impact on the treatment. As an example, preliminary data indicates that the inherent affinity of free ICG for the human ILM is remarkably higher than for the bovine ILM. Since the same could be the case for the ICG-loaded NPs, this example aims to identify potential discrepancies in NP accumulation at the human ILM versus the bovine one. With the regional differences in human ILM morphology in mind, these experiments are performed on both macular and non-macular regions of the posterior retina. In practice, NPs are dripped on top of bovine and human retinal explants and incubated for 1 hour at 4° C. Following a washing step, ICG fluorescence levels are quantitatively compared using the IVIS. Depending on higher or lower NP accumulation as opposed to the bovine situation, we use lower or higher laser energies for ILM perforation, respectively.

### 2) ICG-NP Mediated Photoporation of the Human ILM

ICG-NP treated conventional human retinal explants are irradiated at various laser fluences. Immediately following laser treatment, the retinal surface of intact explants is imaged by Mitotracker staining or samples are processed into cryosections stained for the ILM (Collagen IV) and morphology (H&E). Together, these stainings give insight on ILM integrity and retinal histology, respectively. Furthermore, since these experiments are also performed on macular and non-macular regions of the human retina, the results allow to predict the adaptability of the approach, i.e. how to tailor the treatment parameters to the varying thickness of the ILM ensuring ILM destruction without damaging the inner retina.

### 2. Enhanced Retinal Delivery of Therapeutics by Photoporation

To enhance the delivery of therapeutic carriers into the retina (after injection into the vitreous chamber), experiments are performed as described in Example 3 but with the addition of representative therapeutic carriers instead of polystyrene particles. Co-injection as well as serial injection of the therapeutics and the ICG-NPs is tested. As a quantitative measure of therapeutic (carrier) penetration, the ratio of fluorescence above and below the ILM can be measured in cryosections by confocal microscopy and compared between treated and untreated samples. The following therapeutic(s) (carriers) are tested:

1) AAV2 vectors (GFP-tagged, from Vector Biolabs). Preferably, serotype 2 is used since this is a commonly investigated serotype for intravitreal application.

2) lipid nanoparticles such as MC3-based lipid nanoparticles (about 150 nm, neutral charge);

3) Avastin (bevacizumab, anti-VEGF antibody), routinely intravitreally injected in the clinic for AMD and diabetic macular edema. The antibodies are fluorescently labeled using a commercial antibody labeling kit (Thermo Fischer Scientific).

The results allow to determine which changes might be necessary (laser fluence, NP concentration) to perform the treatment to human tissue; 2) discover for which types of therapeutics ILM perforation can be most beneficial and to which extent.

### Example 5: Efficacy and Safety of ILM Photoporation In Vivo According to Embodiments of the Invention

The ex vivo studies as applied in preceding examples are highly useful to investigate drug delivery methods. Nevertheless, there are in vivo factors at play which are not taken into account ex vivo such as intravitreal clearance. In this example, we look into the in vivo behaviour and toxicity of our NPs as well as the efficacy and safety of the overall ILM photoporation concept in rabbits. The following elements are tested in vivo depending on results of previous examples: 1) co-injection of therapeutic agent with ICG-NPs or injection after laser treatment; 2) one or several ICG-NP types; 3) one or more types of therapeutic agents.

### 1. In Vivo Nanoparticle Distribution and Tolerance Following Intravitreal Injection

Fluorescently labelled ICG-NPs are injected into the vitreous followed by a period of time to allow migration of the particles towards the ILM. Animals are anesthetized for follow-up evaluations over a course of 3 weeks including optical coherence tomography (OCT) and scanning laser ophthalmoscopy (SLO) for morphological assessment of the retina, and electroretinograms (ERG) for functional assessment of the retina. At specific time points (ranging from 1 to 21 days) rabbits are sacrificed followed by eye enucleation, cryosection preparation and subsequent histological staining (H&E) for a detailed view on retinal morphology. At the same time, these sections provide insight on the location of the NPs within the eye in conjunction with the time point at which sufficient ICG-NPs are deposited at the ILM to perform laser treatment.

### 2. Efficacy and Safety of ILM Photoporation In Vivo

As described above, rabbits are intravitreal injected with ICG-NPs (and a therapeutic (carrier) if investigating co-injection) followed by an incubation period. After sufficient accumulation of NPs at the ILM, pulsed laser treatment is performed. Briefly, the setup uses a telescope with two convergent lenses to minimize the size of the focal beam spot on the retina. On top of the eye, a plane-concave lens is placed to reduce aberrations induced by the cornea. Then, an ophthalmoscope for fundus imaging is used to visualize simultaneously the retina and the laser spot. After laser treatment (and IVT injection of therapeutic (carrier) in case of serial injection), the same assays as described above are performed to check on retinal morphology and functionality. In addition, cryosection staining of enucleated eyes is performed to ascertain the integrity of the rabbit ILM as well as the (enhanced) delivery of the therapeutic (vector) within the retina.

The example allows to show the in vivo efficacy and safety of our photoporation method.

1. A a method of photoporation of the inner limiting membrane (ILM) of an eye in a subject, wherein the method comprises:
- administering a dye to a vitreous chamber of the eye of the subject; and
- irradiating at least part of the ILM of the eye of the subject, thereby photoporating at least part of the ILM in the subject.

2. (canceled)

3. A a method of treating a retinal disease or a choroidal disease of an eye in a subject, wherein the method comprises:
- administering a dye to a vitreous chamber of the eye of the subject;
- irradiating at least part of an inner limiting membrane (ILM) of the eye of the subject, thereby photoporating at least part of the ILM: and
- administering a therapeutic agent to the vitreous chamber of the eye of the subject, thereby treating the retinal disease or the choroidal disease in the subject;
or wherein the method comprises:
- administering the dye and the therapeutic agent to the vitreous chamber of an eye of the subject; and
- irradiating at least part of the ILM of the eye of the subject, thereby photoporating at least part of the ILM and treating the retinal disease or the choroidal disease in the subject.

4. (canceled)

5. The method according to claim 3, wherein the dye is a vital dye.

6. The method according to claim 3, wherein the dye is a vital dye selected from the group consisting of: Indocyanine Green (ICG), Trypan Blue (TB), Brilliant Blue (BB), Janus green B (JG), Gentian violet (GV), Bromophenol Blue (BPB), Patent blue (PB), Light Green (LG), Fast Green (FG), Infracyanine Green (IfCG), Methylene blue (MB), Toluidine blue (ToB), Fluorescein Sodium (FS), Rose Bengal (RB), and Rhodamine 6G (R6G); preferably wherein the dye is ICG.

7. The method according to claim 3, wherein the dye is administered at a concentration of about 0.001 mg/ml to about 0.5 mg/ml.

8. The method according to claim 3, wherein the dye is a free dye, an aggregate of the dye, or a crystal of the dye; wherein the dye is conjugated to a further agent; wherein the dye is comprised in a nanoparticle; and/or wherein the dye and the therapeutic agent are comprised in a nanoparticle.

9. The method according to claim 3, wherein the therapeutic agent is a retinal disease drug or a choroidal disease drug.

10. The method according to claim 3, wherein the therapeutic agent is selected from the group consisting of a stem cell, a protein, a polypeptide, a peptide, an antibody, an antibody fragment, an antibody-like protein scaffold, an aptamer, a photoaptamer, a spiegelmer, a peptidomimetic, a gene-editing system, a nucleic acid, and combinations thereof.

11. The method according to claim 3, wherein the therapeutic agent is comprised in a viral vector or in a nanoparticle.

12. The method according to claim 3, 3 wherein the dye and/or the therapeutic agent is administered to the vitreous body by intravitreal injection.

13. The method according to claim 3, wherein the dye and/or the therapeutic agent is administered by injection into the vitreous chamber after removal of the vitreous body.

14. The method according to claim 3, wherein the at least part of the ILM is irradiated with electromagnetic radiation; preferably wherein the at least part of the ILM is irradiated with laser radiation; more preferably wherein the at least part of the ILM is irradiated with pulsed-laser radiation.

15. The method according to claim 3, wherein the retinal disease or the choroidal disease is selected from the group consisting of inherited retinal dystrophies, acquired diseases of the retina or choroid, inflammatory diseases of the retina or choroid, vascular diseases of the retina or choroid, neoplastic diseases of the retina or choroid, ocular traumas, retinal tractional defects, and toxic retinopathy.

16. The method according to claim 3, wherein the retinal disease is selected from the group consisting of macular degeneration, diabetic retinopathy, retinitis pigmentosa, glaucoma, retinoblastoma, and inherited retinal dystrophies.

17. The method according to claim 3, wherein the choroidal disease is selected from the group consisting of central serous chorioretinopathy, polypoidal choroidal vasculopathy, choroidal melanoma, choroidal neovascularization, choroideremia, and choroidal dystrophies.

18. (canceled)

19. The method according to claim 1, wherein one or more of:
- the dye is a vital dye;
- the dye is a vital dye selected from the group consisting of: Indocyanine Green (ICG), Trypan Blue (TB), Brilliant Blue (BB), Janus green B (JG), Gentian violet (GV), Bromophenol Blue (BPB), Patent blue (PB), Light Green (LG), Fast Green (FG), Infracyanine Green (IfCG), Methylene blue (MB), Toluidine blue (ToB), Fluorescein Sodium (FS), Rose Bengal (RB), and Rhodamine 6G (R6G); preferably wherein the dye is ICG;
- the dye is administered at a concentration of about 0.001 mg/ml to about 0.5 mg/ml;
- the dye is a free dye, an aggregate of the dye, or a crystal of the dye;
- the dye is conjugated to a further agent; the dye is comprised in a nanoparticle; and/or the dye and the therapeutic agent are comprised in a nanoparticle;
- the therapeutic agent is a retinal disease drug or a choroidal disease drug;
- the therapeutic agent is selected from the group consisting of a stem cell, a protein, a polypeptide, a peptide, an antibody, an antibody fragment, an antibody-like protein scaffold, an aptamer, a photoaptamer, a spiegelmer, a peptidomimetic, a gene-editing system, a nucleic acid, and combinations thereof;
- the therapeutic agent is comprised in a viral vector or in a nanoparticle;
- the dye and/or the therapeutic agent is administered to the vitreous body by intravitreal injection;
- the dye and/or the therapeutic agent is administered by injection into the vitreous chamber after removal of the vitreous body; and/or
- the at least part of the ILM is irradiated with electromagnetic radiation; preferably wherein the at least part of the ILM is irradiated with laser radiation; more preferably wherein the at least part of the ILM is irradiated with pulsed-laser radiation.

20-21. (canceled)

22. A method for delivering a therapeutic agent to the retina or the choroid of an eye in a subject, the method comprising:
- administering a dye to the vitreous chamber of the eye of the subject;
- irradiating at least part of an inner limiting membrane (ILM)the ILM of the eye of the subject, thereby photoporating at least part of the ILM; and
- administering the therapeutic agent to the vitreous chamber of the eye of the subject;
or the method comprising:
- administering the dye and the therapeutic agent to the vitreous chamber of an eye of the subject; and
- irradiating at least part of the ILM of the eye of the subject, thereby photoporating at least part of the ILM.

23. The method according to claims 22, wherein one or more of:
- the dye is a vital dye;
- the dye is a vital dye selected from the group consisting of: Indocyanine Green (ICG), Trypan Blue (TB), Brilliant Blue (BB), Janus green B (JG), Gentian violet (GV), Bromophenol Blue (BPB), Patent blue (PB), Light Green (LG), Fast Green (FG), Infracyanine Green (IfCG), Methylene blue (MB), Toluidine blue (ToB), Fluorescein Sodium (FS), Rose Bengal (RB), and Rhodamine 6G (R6G); preferably wherein the dye is ICG;
- the dye is administered at a concentration of about 0.001 mg/ml to about 0.5 mg/ml;
- the dye is a free dye, an aggregate of the dye, or a crystal of the dye;
- the dye is conjugated to a further agent; the dye is comprised in a nanoparticle; and/or the dye and the therapeutic agent are comprised in a nanoparticle;
- the therapeutic agent is a retinal disease drug or a choroidal disease drug;
- the therapeutic agent is selected from the group consisting of a stem cell, a protein, a polypeptide, a peptide, an antibody, an antibody fragment, an antibody-like protein scaffold, an aptamer, a photoaptamer, a spiegelmer, a peptidomimetic, a gene-editing system, a nucleic acid, and combinations thereof;
- the therapeutic agent is comprised in a viral vector or in a nanoparticle;
- the dye and/or the therapeutic agent is administered to the vitreous body by intravitreal injection;
- the dye and/or the therapeutic agent is administered by injection into the vitreous chamber after removal of the vitreous body;
- the at least part of the ILM is irradiated with electromagnetic radiation; preferably wherein the at least part of the ILM is irradiated with laser radiation; more preferably wherein the at least part of the ILM is irradiated with pulsed-laser radiation;
- the retinal disease or the choroidal disease is selected from the group consisting of inherited retinal dystrophies, acquired diseases of the retina or choroid, inflammatory diseases of the retina or choroid, vascular diseases of the retina or choroid, neoplastic diseases of the retina or choroid, ocular traumas, retinal tractional defects; and toxic retinopathy;
- the retinal disease is selected from the group consisting of macular degeneration, diabetic retinopathy, retinitis pigmentosa, glaucoma, retinoblastoma, and inherited retinal dystrophies; and/or
- the choroidal disease is selected from the group consisting of central serous chorioretinopathy, polypoidal choroidal vasculopathy, choroidal melanoma, choroidal neovascularization, choroideremia, and choroidal dystrophies.
